(19) United States
(12) Patent Application Publication
(10) Pub. No.: US 2023/0408498 A1
(43) Pub. Date: Dec. 21, 2023

(54) COMPOSITIONS AND METHODS INVOLVING ANTIBODY CONSTRUCTS

(71) Applicants: CZ BIOHUB SF, LLC, San Francisco, CA (US); The Board of Trustees of the Leland Stanford Junior University, Stanford, CA (US)

(72) Inventors: Jing Pan, Stanford, CA (US); Ian Andrew Paul Thompson, Stanford, CA (US); Brandon Wilson, Stanford, CA (US); Hyongsok Tom Soh, Palo Alto, CA (US); Nicolo Maganzini, Stanford, CA (US)

(21) Appl. No.: 18/248,436

(22) PCT Filed: Oct. 12, 2021
(86) PCT No.: PCT/US2021/054522

§ 371 Date: Apr. 10, 2023

Related U.S. Application Data
(60) Provisional application No. 63/090,918, filed on Oct. 13, 2020.

(51) Int. Cl. G01N 33/542; G01N 33/68

(52) U.S. Cl. CPC G01N 33/542; G01N 2333/7158; G01N 2458/10; G01N 33/6863

(57) ABSTRACT

The disclosure provides antibody constructs that can reversibly bind to biological or chemical target analytes. Upon target analyte binding, the antibody construct can change its conformational state to produce a detectable readout. An antibody construct can be a single-antibody construct or a dual-antibody construct.

### CROSS-REFERENCES TO RELATED APPLICATIONS

This application claims priority to U.S. Provisional Application No. 63/090,918, filed Oct. 13, 2020, the disclosure of which is hereby incorporated by reference in its entirety for all purposes.

### BACKGROUND OF THE INVENTION

Molecular switches that change their conformation upon target analyte binding offer powerful capabilities for biotechnology and synthetic biology.

### BRIEF SUMMARY OF THE INVENTION

In one aspect, the disclosure features an antibody construct comprising: an antibody or a binding fragment thereof comprising a first label, wherein the antibody or the binding fragment thereof is linked to a blocking analyte and a second label via a linker, wherein: in the absence of a target analyte, the blocking analyte binds to the antibody or the binding fragment thereof, and in the presence of the target analyte, the target analyte competes with the blocking analyte for binding to the antibody or the binding fragment thereof, and the first and second labels interact to generate a detectable readout that changes depending on whether the blocking analyte is bound by the antibody or the binding fragment thereof, or is not bound by the antibody or the binding fragment thereof.

In some embodiments, the second label is conjugated to the linker. In some embodiments, the second label is conjugated to the blocking analyte. In some embodiments, the first label is a fluorophore and the second label is a quencher. In some embodiments, the first label is a quencher and the second label is a fluorophore. In some embodiments, the first label is a donor fluorophore and the second label is an acceptor fluorophore. In some embodiments, the first label is an acceptor fluorophore and the second label is a donor fluorophore.

In some embodiments, the detectable readout is an optical signal, an electrical signal, an electrochemical signal, a nuclear magnetic resonance signal, or a biological signal. In some embodiments, the optical signal is a fluorescent signal.

In some embodiments, binding of the target analyte to the antibody or the binding fragment thereof increases or decreases the detectable readout.

Also provided is an antibody construct comprising: an antibody or a binding fragment thereof comprising a redox reporter, wherein the antibody or the binding fragment thereof is linked to a blocking analyte and a sensing electrode; wherein: in the absence of a target analyte, the blocking analyte binds to the antibody or the binding fragment thereof, and in the presence of the target analyte, the target analyte competes with the blocking analyte for binding to the antibody or the binding fragment thereof, and the redox reporter and the sensing electrode interact to generate an electrical signal that changes depending on whether the blocking analyte is bound by the antibody or the binding fragment thereof, or is not bound by the antibody or the binding fragment thereof.

In some embodiments, the antibody or the binding fragment thereof is linked to the blocking analyte and the sensing electrode via a linker. In some embodiments, the antibody or the binding fragment thereof is linked to the blocking analyte by a first linker and the antibody or the binding fragment thereof is linked to the sensing electrode via a second linker. In some embodiments, the antibody or the binding fragment thereof is linked to the sensing electrode via a gold-thiol bond. In some embodiments, the sensing electrode is a gold electrode.

In some embodiments, the linker is a double-stranded polynucleotide. In some embodiments, the linker is a single-stranded polynucleotide. In some embodiments, the linker is a partially double-stranded polynucleotide comprising at least one unpaired nucleotide. In some embodiments, the partially double-stranded polynucleotide comprises between 1 and 30 unpaired nucleotides. In some embodiments, the linker is a partially double-stranded polynucleotide, and wherein at least 1% of the length of the linker comprises unpaired nucleotide(s). In some embodiments, between 1% and 50% of the length of the linker comprises unpaired nucleotide(s).

In some embodiments, the unpaired nucleotides are located at the 5′ terminus or the 3′ terminus of the linker.

In some embodiments, the length of the linker is at least 10 nucleotides. In some embodiments, the length of the linker is between 10 and 100 nucleotides.

In some embodiments, the linker comprises one or more mismatched nucleotides.

In some embodiments, the antibody is covalently conjugated to the linker via an Fc region of the antibody.

In some embodiments, the blocking analyte and the target analyte are the same.

In some embodiments, blocking analyte is a structural analog of the target analyte.

In some embodiments, a binding affinity of the antibody to the target analyte is between 90% and 110% of a binding affinity of the antibody to the blocking analyte. In some embodiments, the antibody has a higher binding affinity for the target analyte than for the blocking analyte. In some embodiments, the antibody has a lower binding affinity for the target analyte than for the blocking analyte.

In some of the embodiments described herein, the blocking analyte can bind to the antibody in the antibody construct independent of the antibody's cognate antigen type. In particular embodiments, the blocking analyte is Protein M, Protein A, Protein G, or Protein L (e.g., Protein M).

Also provided is an antibody construct comprising: (a) two detecting strands, wherein a first detecting strand comprises a first antibody or a binding fragment thereof, and a second detecting strand comprises a second antibody or a binding fragment thereof; and (b) a first label and a second label, wherein the detecting strands or portions thereof are complementary and hybridize to each other or one or more scaffold strands, and wherein in the presence of the target analyte, the first antibody and the second antibody bind to two different epitopes on the target analyte, and the first and second labels interact with each other to generate a detectable readout compared to when there is an absence of the target analyte.

In some embodiments, the two detecting strands hybridize to a scaffold strand, thereby linking the two detecting strands. In some embodiments, the two detecting strands hybridize to a single scaffold strand. In some embodiments, two detecting strands hybridize to separate scaffold strands which scaffold strands hybridize to each other.

In some embodiments, the two detecting strands hybridize to each other, thereby linking the two detecting strands.

In some embodiments, the first label is linked to the first antibody and the second label is linked to the second antibody. In some embodiments, the first label and/or second label is linked to a scaffold strand. In some embodiments, the first label is linked to a first label oligonucleotide that is hybridized to a scaffold strand and second label is linked to a second label oligonucleotide that is hybridized to a scaffold strand. In some embodiments, the first label is linked to a first scaffold strand and the second label is linked to a second scaffold strand. In some embodiments, in the presence of the target analyte, a portion of the first scaffold strand and a portion of the second scaffold strand hybridize to each other.

In some embodiments, the antibody construct comprises two scaffold strands, wherein a first scaffold strand and a second scaffold strand are complementary and hybridize to each other. In some embodiments, the first detecting strand or a portion thereof, the second detecting strand or a portion thereof, and the second scaffold strand or a portion thereof are complementary and hybridize to the first scaffold strand in a linear order with the second scaffold strand between the first and second detecting strands. In some embodiments, the first scaffold strand is at least 100 nucleotides long In some embodiments, the first scaffold strand is between 100 and 200 nucleotides long.

In some embodiments, the second scaffold strand is at least 20 nucleotides long. In some embodiments, the second scaffold strand is between 20 and 180 nucleotides long. In some embodiments, each of the first and second detecting strands is at least 20 nucleotides long. In some embodiments, each of the first and second detecting strands is between 20 and 40 nucleotides long.

In some embodiments, the antibody construct comprises three or more scaffold strands. In some embodiments, each of the three or more scaffold strands is independently at least 100 nucleotides long. In some embodiments, each of the three or more scaffold strands is independent between 100 and 200 nucleotides long. In some embodiments, pairs of the scaffold strands or portions thereof are complementary and hybridize to each other.

In some embodiments, the first antibody is covalently conjugated to the first detecting strand via an Fc region of the first antibody, and wherein the second antibody is covalently conjugated to the second detecting strand via an Fc region of the second antibody.

In some embodiments, the scaffold strands comprise one or more mismatched nucleotides.

In some embodiments, the detectable readout is an optical signal, an electrical signal, an electrochemical signal, a nuclear magnetic resonance signal, or a biological signal. In some embodiments, the optical signal is a fluorescent signal.

In some embodiments, the binding of the target analyte to the first and second antibodies increases or decreases the detectable readout.

Also provided is a method of detecting a target analyte in a sample, comprising: (1) contacting the sample with an antibody construct as described above or elsewhere herein; and (2) measuring binding of the antibody construct to the target analyte using a detectable readout from the antibody construct.

In some embodiments, the detectable readout is an optical signal, an electrical signal, an electrochemical signal, a nuclear magnetic resonance signal, or a biological signal. In some embodiments, the optical signal is a fluorescent signal.

In some embodiments, the sample is a biological sample.

Also provided is a method of adjusting kinetics and/or effective binding affinity of an antibody construct, comprising: (1) generating an antibody construct as described above or elsewhere herein; (2) measuring binding of the antibody construct to a target analyte; (3) changing one or more components of the antibody construct; (4) re measure binding of the antibody construct to the target analyte; and(5) optionally repeat steps (3) and (4) until the desired kinetics and/or effective binding affinity of the antibody construct is reached.

In some embodiments, step (3) comprises increasing or decreasing the length of the linker. In some embodiments, step (3) comprises introducing one or more mismatched nucleotides into the linker. In some embodiments, step (3) comprises changing the identity of the blocking analyte. In some embodiments, step (3) comprises increasing or decreasing the length of one or more scaffold strands. In some embodiments, step (3) comprises introducing one or more mismatched nucleotides into one or more scaffold strands. In some embodiments, step (3) comprises introducing a structural motif into the linker.

In some embodiments, the structural motif is a DNA/RNA duplex, a polynucleotide hairpin, a G-quadruplex, or an i-motif.

### BRIEF DESCRIPTION OF THE DRAWINGS

FIGS. 1A-1D depict a schematic of one embodiment of the antibody-switch construct using a fluorescent reporting mechanism to signal target binding. Solution-based testing of this construct (FIG. 1B), assembled with a biotin molecule acting as the “bait” shows increases in fluorescence upon addition of free biotin, suggesting switching and signaling. Surface-tethered bead-based measurements of concentration-dependent switching (FIGS. 1C and 1D) show the fluorescence is concentration-dependent, and that the construct functions both in buffer and more complex media (1% serum).

FIGS. 2A-2E depict surface-tethered microscopy-based measurements of switch activation (FIG. 2B) confirm observable fluorescent switching from target binding. Concentration-dependent switching kinetics (FIGS. 2C-2E) show the kinetic response of this construct can be easily observed for both on- and off-switching.

FIG. 3 depicts changing the secondary structure design of the scaffold by varying the portion of the scaffold that assumes a duplex/single stranded structure, which in turn varies the scaffold stiffness and allows tuning of the affinity. Results for varied scaffold strandedness from 0%-100% double stranded confirms broad tunability of affinity.

FIG. 4 depicts changing the length of the scaffold affects the effective concentration of the bait molecule and allows tuning of the affinity. Results for varied double stranded scaffold lengths from 20 bp to 80 bp shows that a variety of construct designs function, and that the length can impart effects on the switch affinity.

FIGS. 5A-5C depict changing the identity of the bait on the blocking strand from the antibody's cognate antigen to a mismatched molecular analog allows tuning of the affinity. Here we show that by switching the biotin bait for a desthiobiotin molecule with lower affinity for the antibody than the biotin target, we can increase the switch's effective affinity.

FIG. 6 depicts a general synthesis workflow for synthesizing the single-antibody switch constructs described herein. The process is designed to be modular, and relies on conjugating the bait of choice to the DNA scaffold, then assembling the remaining construct and reporters through attachment of DNA to the Fc region of the antibody.

FIG. 7 depicts a schematic of one embodiment of the antibody-switch construct using two antibodies to bind different epitopes of the target to activate fluorescent reporting. Proof of concept for a TNF-alpha sensing embodiment of this switch demonstrated using two TNF-alpha binding monoclonal antibodies, and the construct was measured on a fluorescent microscopy-based system. The results for both a fluorophore-quencher (middle) and a donor-acceptor (right) system confirm target dependent switching and fluorescent signaling for this construct in response to addition of TNF-alpha.

FIG. 8 depicts a schematic of an electrochemical readout scheme for sensing using the antibody switch by incorporating redox reporting moieties conjugated onto the antibody (detecting strand). Data for real-time measurement using this system shows target-responsiveness through electrochemical readout.

FIG. 9 depicts an embodiment similar to that depicted in FIG. 8 but with a difference that a separate linker attaches the entire construct to the electrode. This linker can be a polynucleotide linker or a small chemical bi-functional linker (e.g. an NHS-thiol) and need not have functional properties besides covalently fixing the antibody to the surface.

FIG. 10 depicts changing the length of the dual antibody switch scaffold by including one, two or three separate scaffold strands (and their complementary strands) of variable length, giving rise to a scaffold length between 150 bp and 540 bp. Changing the length and complementarity of the complementary strands leads to varying degrees of scaffold flexibility. In this case, there are no label strands, as the labels are conjugated with the antibodies.

FIG. 11 depicts a synthesis scheme for achieving DNA crosslinking of a vancomycin blocking analyte, and the resulting data for fluorescent switching and reversibility.

FIGS. 12A-12C depict results from testing the vancomycin-responsive antibody switch construct of FIG. 11 fluorescent response by adding free vancomycin.

FIGS. 13A and 13B depict a surface-tethered bead-based reversibility assay showing that the biotin-responsive single antibody switch can be used to continuously measure changing analyte concentrations through many cycles of increasing or decreasing concentrations.

FIGS. 14A and 14B depict a surface-tethered bead-based assay showing that the biotin-responsive single antibody switch responds to changing target concentration with fast temporal resolution that enables rapid measurement.

FIG. 15 depicts another embodiment of the antibody-switch construct employing a single antibody and achieving target-responsive molecular switching in response to a small molecule steroid, digoxigenin (DIG).

FIG. 16 depicts a surface-tethered bead-based reversibility assay showing that the digoxigenin-responsive single antibody switch can be used to continuously measure changing analyte concentrations.

FIGS. 17A and 17B depict the general synthesis workflow for synthesizing the scaffold and blocking strand for the digoxigenin-responsive antibody switch.

FIGS. 18A-18C depict MDAC. a) The MDAC is a 3-component system: two dye-labeled monoclonal antibodies linked by a DNA scaffold. In the presence of target, the antibodies bind to different epitopes on the protein, leading to increased dye proximity and FRET. The FRET ratio is used to quantify the fraction of bound MDAC structures. b) Schematic of synthesis procedure for antibody-dye-oligo conjugates, followed by assembly of MDAC structure on streptavidin beads. c) MDAC bead assembly was validated via assembly on streptavidin beads and imaging on a flow cytometer: bare beads—background fluorescence of streptavidin beads; no Ab1 control—omitting Ab 1 in the assembly procedure shows no non-specific binding of scaffold or Ab2 on the beads; Ab1 only—immobilization of Ab1 on beads surface leads to increase in fluorescence; no scaffold control—omitting the DNA scaffold from the full assembly leads to similar fluorescence to Ab1 only, indicating no non-specific interactions between the two antibodies; full assembly—assembly protocol for MDAC shows increase in both antibody fluorescence channels. The resulting starting FRET Ratio is consistent and reproducible.

FIGS. 19A-19D depict MDAC binding model and thermodynamics. a) MDAC five-state binding model and equilibrium relationships. b) Determination of kₒₙ as slope of best linear fit to Eq. 2 (error bars on k obs values are present but not visible), and calculation of K_{D} values for mAb1 and mAb11. c) Fluorescence measurements (Green—donor excitation and emission; FRET—donor excitation and acceptor emission; Red—acceptor excitation and emission) of bead-immobilized MDAC challenged with TNFa concentrations carried out on a cytometer. Error bars represent standard deviation from 3 replicates. d) FRET ratio is computed from the Green and FRET measurements. Eq. 1 is fit to the ratio data and plotted alongside it. Error bars represent standard deviation of FRET ratio calculation.

FIGS. 20A-20D depict MDAC rapid binding in serum, nuclease degradation. a) Cytometry measurements (Green—donor excitation and emission; FRET—donor excitation and acceptor emission; Red—acceptor excitation and emission) of bead-immobilized MDAC incubated for 30 minutes with TNFa concentrations in buffer and chicken serum. Error bars represent standard deviation from 3 replicates. b) Calculated FRET ratio of MDAC 30-minute binding signal in buffer and serum. Error bars represent the standard deviation of 3 replicates. c) Degradation of MDAC DNA scaffold through nuclease activity in serum. The distal antibody is cleaved off the surface of the bead leading to a decrease in green fluorescence. d) FRET Radio degradation due to nuclease activity from a 30-minute serum incubation. Engineered nuclease resistance in the DNA components of MDAC can be used to reduce degradation.

FIGS. 21A-21E depict synthesis and characterization of MCP-1 MDAC. a) Assembly of MDAC with MCP-1 antibodies, and associated controls. Error bars represent standard deviation of fluorescence from three different bead assemblies. Decreased red fluorescence compared to TNFa MDAC is due to the use of Atto 643, rather than Alexa Fluor 647, as the red acceptor dye. b) Fluorescence measurements (Green—donor excitation and emission; FRET—donor excitation and acceptor emission; Red—acceptor excitation and emission) of bead-immobilized MDAC challenged with MCP-1 concentrations and incubated overnight. Error bars represent standard deviation from 3 replicates. c) FRET ratio is computed from the Green and FRET measurements. Eq. 1 is fit to the ratio data and plotted alongside it. Error bars (not visible) represent standard deviation of FRET ratio calculation across three measurements. d) Fluorescence measurements of bead-immobilized MDAC incubated for 30 minutes with MCP-1 concentrations in buffer and chicken serum. Error bars (not visible) represent standard deviation from 3 replicates. e) Calculated FRET ratio of MDAC 30-minute binding signal in buffer and serum. Error bars (not visible) represent the standard deviation of 3 replicates.

FIGS. 22A-22C depict MCP-1 binding response on optical fiber. a) Optical fiber surfaces were prepared with MCP-1 MDAC and exposed to different concentrations of MCP-1 in buffer. MCP-1 MDAC showed clearly discernible binding kinetics in the relevant concentration range. b) Exponential curves were fit to the data in a) and the rates were plotted vs MCP-1 concentration. c) Data in a) was re-organized and plotted vs concentration for different incubation times.

FIG. 23 depicts Instant ELISA with MDAC. The Instant ELISA platform consists of a fiber optic probe surface prepared with MDAC constructs. When the probe is dipped in a blood sample, MDAC molecules will bind to the protein target and structure-switch, generating a FRET fluorescence signal. The MDAC emission is collected by the fiber and measured by an optical detection backend. The protein concentration is then deduced from the binding kinetics of MDAC.

### DETAILED DESCRIPTION OF THE INVENTION

### I. Introduction

The disclosure provides antibody constructs that can be used as programmable affinity reagents for applications in, for example, molecular diagnostics, biomedical imaging, point-of-care devices, and home-use tests. Depending on the assay format, the antibody construct can enable reversible, rapid, quantitative, sensitive, and multiplexed biomolecule detection in complex biological samples.

The antibody constructs described herein can bind specifically to biological or chemical target analytes (e.g., small molecules, peptides, drugs, glycans, proteins, and DNA, etc.). Upon target analyte binding, the antibody construct changes its conformational state to produce a detectable readout. As described herein, many configurations of the antibody constructs are possible. For example, an antibody construct can be a single antibody construct or a double antibody construct. An antibody construct can have two conformational states, i.e. a bi-state molecular switch. The population distribution between the two states, as well as the transition kinetics between the states, can be programmed by designing and tailoring various components in the antibody constructs described herein, such as the linker, the scaffold strands, and the detecting strands. The conformation of the antibody construct can be dynamically modulated by environmental factors such as the presence of the target analytes. The changes in population distribution between the conformational states can be transduced to detectable readouts.

In some embodiments of a single antibody construct, the antibody construct can contain an antibody and a blocking analyte. The presence of target analyte can disrupt the interaction between the antibody and blocking analyte and switch the construct to the “ON” state. In some embodiments of the double antibody construct, the antibody construct can contain two antibodies targeting different epitopes on the same target analyte. The present of target analyte can induce a sandwich complex formation and change the construct conformation.

### II. Definitions

As used herein, the term “antibody construct” refers to a molecular structure that changes its conformation upon binding to a target analyte. In one example, an antibody construct can contain an antibody comprising a first label, in which the antibody is linked to a blocking analyte and a second label by way of a linker. In another example, an antibody construct can contain at least two scaffold strands, two detecting strands, and two labels.

As used herein, the term “linker” refers to a linkage between two elements, e.g., an antibody and a blocking analyte in an antibody construct. A linker can be a polymer, e.g., a polynucleotide or a polypeptide, that can provide space and/or flexibility between an antibody and a blocking analyte in the antibody construct.

As used herein, the term “label” refers to an agent that can produce a measurable signal or block a measurable signal. For example, a label can be a fluorophore or a quencher. In some embodiments, when two labels interact to form a Fluorescence Resonance Energy Transfer (FRET) pair, a label can be a donor fluorophore or an acceptor fluorophore. In other embodiments, a label can be a redox reporter.

As used herein, the term “scaffold strand” refers to a component of the antibody construct (e.g., a dual-antibody construct) that does not have an antibody linked to it. In some embodiments, a scaffold strand can have one or more labels linked to the scaffold strand. In some embodiments, the scaffold strand can be a polynucleotide. In some embodiments, the scaffold strand can contain a mixture of nucleotides, amino acids, and small organic molecules. In certain embodiments, the scaffold strand can contain small organic molecules (e.g., polyethylene glycol (PEG) and/or polypropylene glycol (PPG)), in addition to nucleotides. The scaffold strand can be designed to provide flexibility to the conformation of the antibody construct. In some embodiments, one or more scaffold strands or portions thereof in an antibody construct are complementary and can hybridize to one or more detecting strands or portions thereof to form a double-stranded polynucleotide in an antibody construct.

As used herein, the term “detecting strand” refers to a component of the antibody construct that comprises an antibody and can recognize and bind to a target analyte via the affinity between the antibody conjugated to the detecting strand and the target analyte. In some embodiments, the full length or only a portion of the detecting strand is hybridized to one or more scaffold strands or portions thereof. In some embodiments, the antibody is conjugated to a terminus of the detecting strand. In other embodiments, the antibody is conjugated to an internal region of the detecting strand. In some embodiments, the detecting strand can be a polynucleotide. In some embodiments, the detecting strand can contain a mixture of nucleotides, amino acids, and small organic molecules. In certain embodiments, the detecting strand can contain small organic molecules (e.g., polyethylene glycol (PEG) and/or polypropylene glycol (PPG)), in addition to nucleotides.

As used herein, the term “antibody” refers to a protein functionally defined as a binding protein and structurally defined as comprising an amino acid sequence that is recognized by one of skill as being derived from a variable region of an immunoglobulin encoding gene. The term encompasses intact polyclonal antibodies, intact monoclonal antibodies, single chain antibodies, multispecific antibodies such as bispecific antibodies, monospecific antibodies, monovalent antibodies, chimeric antibodies, humanized antibodies, and human antibodies. The term “antibody,” as used herein, also includes antibody fragments that retain binding specificity, including but not limited to Fab, F(ab′)₂, Fv, scFv, and bivalent scFv. An antibody can consist of one or more polypeptides substantially encoded by immunoglobulin genes or fragments of immunoglobulin genes. The recognized immunoglobulin genes include the kappa, lambda, alpha, gamma, delta, epsilon, and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. The term “antibody,” as used herein, also includes antibody mimetic proteins, for example affibodies, DARPins, and nanobodies.

An exemplary antibody structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms “variable light chain” (VL) and “variable heavy chain” (VH) refer to these light and heavy chains, respectively.

As used herein, the term “detectable readout” refers to a measurable signal or the absence of any measurable signal. A measurable signal can be a physical and/or chemical signal, e.g., a fluorescent signal or an electrochemical signal. For example, a detectable readout can be a fluorescent signal produced by a fluorophore. A detectable readout can also be the disappearance of a fluorescent signal when the fluorophore is interacting with a quencher, which quenches the fluorescent signal.

As used herein, the term “fluorophore” refers to a compound, e.g., a small molecule or a protein, which when excited by exposure to a particular wavelength of light, emits light at a different wavelength. Fluorophores can be characterized in terms of their emission profile, or “color.” For example, green fluorophores (e.g., green fluorescent protein (GFP), Cy3, FITC, and Oregon Green) are generally characterized by their emission at wavelengths in the range of 510-550 nm. Red fluorophores (e.g., red fluorescent protein (RFP), Texas Red, Cy5, and tetramethylrhodamine) are generally characterized by their emission at wavelengths in the range of 590-690 nm.

As used herein, the term “target analyte” refers to a molecule that can be recognized and bound by the antibody in the antibody construct. A target analyte can be a small molecule (e.g., a small organic molecule), a protein, a glycan, a drug (for example but not limited to vancomycin), a peptide, or a nucleic acid (e.g., DNA or RNA).

As used herein, the term “blocking analyte” refers to a molecule that can be recognized and bound by the antibody in the antibody construct and is linked to the antibody by way of a linker. In some embodiments, the blocking analyte can be the same as the target analyte. In other embodiments, the blocking analyte can be a structural analog of the target analyte, which means that the blocking analyte can have a structure similar to that of the target analyte but differing from the target analyte in certain components of the structure, such as one or more atoms or functional groups. In yet other embodiments, a blocking analyte can be a molecule that can bind to the antibody in the antibody construct independent of the antibody's cognate antigen type.

As used herein, the term “hybridize” or “hybridization” refers to the annealing of nucleobases, nucleosides, or nucleotides of a polynucleotide to the nucleobases, nucleosides, or nucleotides at the corresponding positions of another polynucleotide. In some embodiments, hybridization can happen through hydrogen bonding interactions that occur between nucleobases, nucleosides, or nucleotides. The hydrogen bonding interactions may be Watson-Crick hydrogen bonding or Hoogsteen or reverse Hoogsteen hydrogen bonding. In some embodiments, nucleobases, nucleosides, or nucleotides of a polynucleotide can hybridize to the nucleobases, nucleosides, or nucleotides at the corresponding positions of another polynucleotide via partial complementarity or complete complementarity.

As used herein, the term “complementary” or “complementarity” refers to the capacity for base pairing via Watson-Crick hydrogen bonding interactions between nucleobases, nucleosides, or nucleotides of a polynucleotide to the nucleobases, nucleosides, or nucleotides at the corresponding positions of another polynucleotide. Examples of complementary nucleobase pairs include, but are not limited to, adenine and thymine, cytosine and guanine, and adenine and uracil, which all pair through the formation of hydrogen bonds. In some embodiments, a polynucleotide or a portion thereof in the antibody construct can have “complete complementarity” to another polynucleotide or a portion thereof, which means that all of the nucleotides the first polynucleotide that form the hybridization are complementary to the corresponding nucleotides in the other polynucleotide. In other embodiments, a polynucleotide or a portion thereof in the antibody construct can have “partial complementarity” to the other polynucleotide or a portion thereof, which means that at least one of the nucleotides in the first polynucleotide does not form Watson-Crick hydrogen bonding with the nucleotide at the corresponding position of the other polynucleotide. Two polynucleotides having “partial complementarity” can contain at least one mismatched nucleotide and can be referred to as a partially double-stranded polynucleotide. A “degree of complementarity” or “% complementarity” refers to a percentage of nucleobases, nucleosides, or nucleotides of a polynucleotide that form Watson-Crick hydrogen bonding interactions with the nucleobases, nucleosides, or nucleotides at the corresponding positions of the other polynucleotide. A “perfect degree of complementarity” or “100% complementarity” refers to that all of the of nucleobases, nucleosides, or nucleotides in the first polynucleotide or a portion thereof that form the hybridization with the other polynucleotide or a portion thereof are in Watson-Crick hydrogen bonding interactions with the nucleobases, nucleosides, or nucleotides at the corresponding positions of the other polynucleotide.

As used herein, the term “mismatched nucleotide” refers to a nucleotide at a specific position in the polynucleotide that does not engage in Watson-Crick base pairing with a nucleotide at the corresponding position in another polynucleotide when the two polynucleotides hybridize.

### III. Antibody Constructs

The disclosure provides antibody constructs that can change their conformation upon binding to a target analyte. An antibody construct can contain one antibody conjugated to a first label, and the antibody can be further linked to a blocking analyte and a second label by way of a linker. In another example, an antibody construct can be a dual-antibody construct that contains two antibodies linked to detecting strands. The detecting strands can further hybridize to one or more scaffold strands to form an antibody construct having the structure of a double-stranded or partially double-stranded polynucleotide.

### Single-Antibody Construct

An antibody construct disclosed herein can be a single-antibody construct. In one example, a single-antibody construct comprises: an antibody or a binding fragment thereof comprising a first label, wherein the antibody or the binding fragment thereof is linked to a blocking analyte and a second label via a linker. In a single-antibody construct, in the absence of a target analyte, the blocking analyte binds to the antibody or the binding fragment thereof, and in the presence of the target analyte, the target analyte competes with the blocking analyte for binding to the antibody or the binding fragment thereof. Further, the first and second labels in the single-antibody construct can interact to generate a detectable readout that changes depending on whether the blocking analyte is bound by the antibody or the binding fragment thereof, or is not bound by the antibody or the binding fragment thereof.

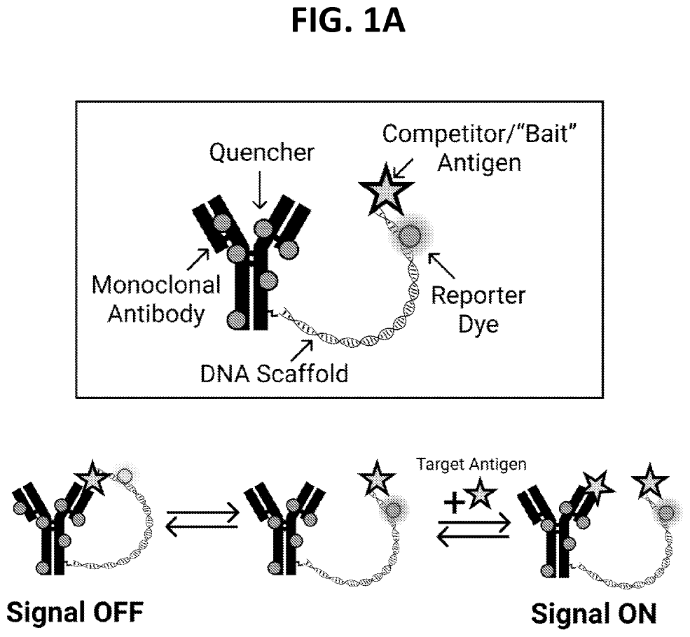

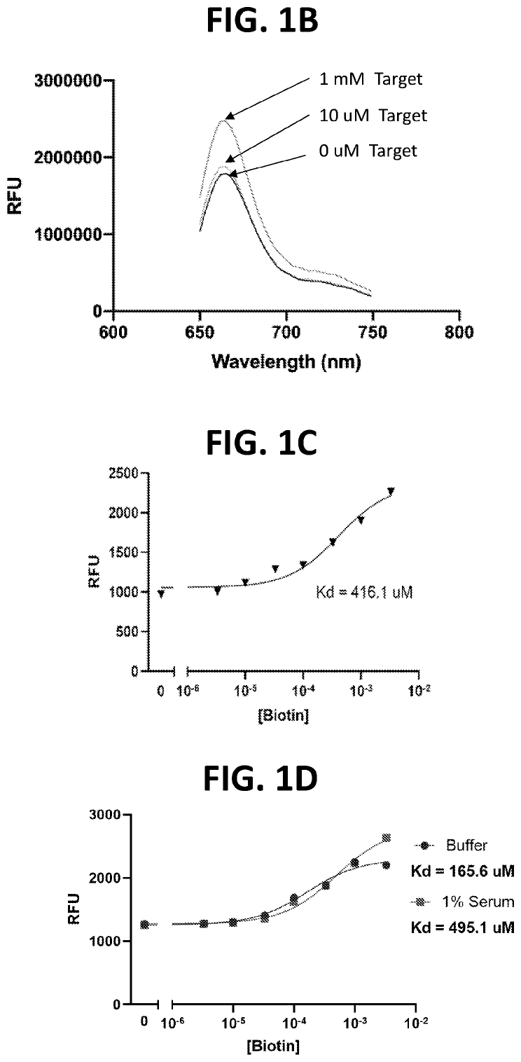

In a single-antibody construct, in some embodiments, the first label can be conjugated to the antibody and the second label can be conjugated to the linker. (see, e.g., FIG. 1). In other embodiments, the second label can be conjugated to the blocking analyte.

The first and second labels are selected so that signal from the labels change based on their proximity, namely such that there is a measurable difference in signal when the antibody binds to the blocking analyte compared to a conformation when the blocking analyte is displaced by the target analyte. In certain embodiments, the first label can be a fluorophore and the second label can be a quencher. In certain embodiments, the first label can be a quencher and the second label can be a fluorophore. In cases where one of the two labels is a quencher, in the absence of a target analyte, the blocking analyte binds to the antibody, bringing the first and second labels in proximity, such that the fluorescent signal from one label is quenched by the other label and no signal is detected. In the presence of a target analyte, the target analyte can bind to the antibody, such that the first and second labels are not in proximity to each other and the fluorescent signal from one of the labels is detected.

In other embodiments, the first label can be a donor fluorophore and the second label can be an acceptor fluorophore to form a FRET pair. In other embodiments, the first label can be an acceptor fluorophore and the second label can be a donor fluorophore to form a FRET pair. Examples of fluorophores that form FRET pairs are described in detail further below. In the absence of a target analyte, the blocking analyte binds to the antibody, bringing the first and second labels in proximity, such that the emission spectrum of the donor fluorophore partially overlaps the excitation spectrum of the acceptor fluorophore and a FRET signal is produced. In the presence of a target analyte, the target analyte can bind to the antibody, such that the first and second labels are not in proximity to each other and no FRET signal is produced.

The detectable readout generated from the single-antibody construct can be an optical signal (e.g., a fluorescent signal), an electrical signal, an electrochemical signal, a nuclear magnetic resonance signal, or a biological signal. The binding of the target analyte to the antibody or the binding fragment thereof can increase or decrease the detectable readout.

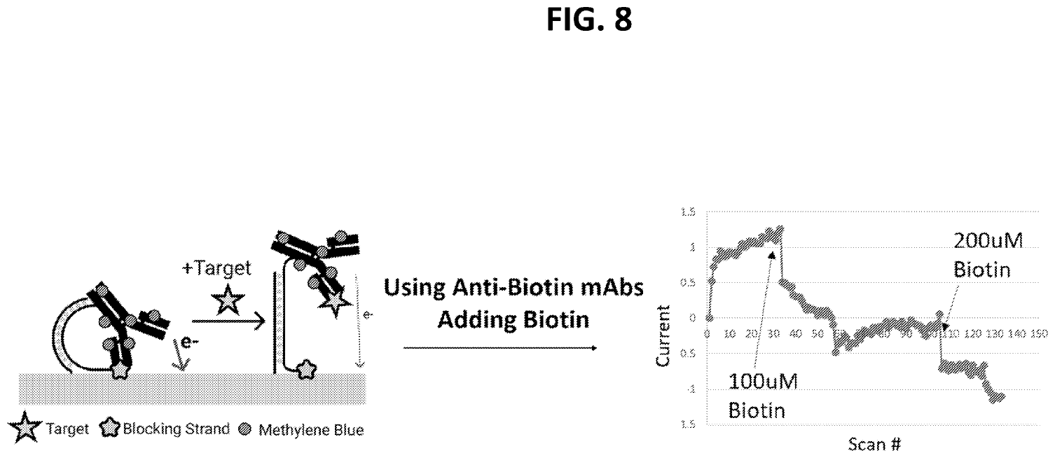

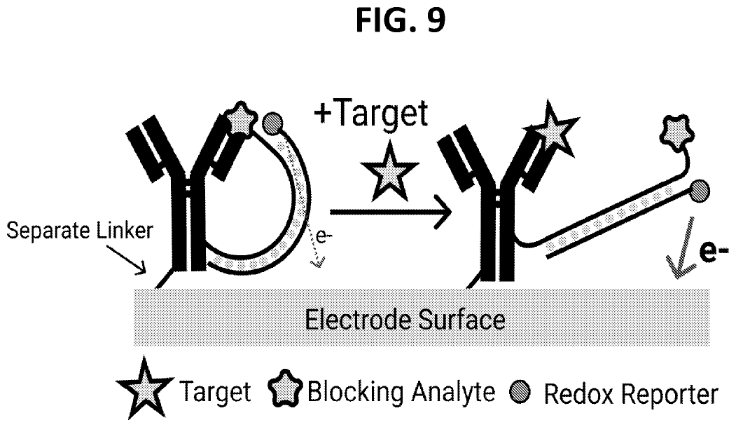

In another example of a single-antibody construct, an antibody or a binding fragment thereof can be conjugated to a redox reporter and further linked to a blocking analyte and a sensing electrode via a linker. See, e.g., FIGS. 8 and 9. In the absence of a target analyte, the blocking analyte can bind to the antibody or the binding fragment thereof, and in the presence of the target analyte, the target analyte can compete with the blocking analyte for binding to the antibody or the binding fragment thereof. The redox reporter and the sensing electrode can interact to generate an electrical signal that changes depending on whether the blocking analyte is bound by the antibody or the binding fragment thereof, or is not bound by the antibody or the binding fragment thereof. In some embodiments, the antibody is linked to the blocking analyte and sensing electrode through the same linker. See, e.g., FIG. 8. In other embodiments, the antibody is linked to the blocking analyte and sensing electrode through the different linkers (e.g., a first and a second linker). See, e.g., FIG. 9.

As shown in FIG. 8, in some embodiments, a first polynucleotide strand is linked to the sensing electrode and the antibody is linked to a second polynucleotide strand that hybridizes to the first strand. In some embodiments, the antibody is linked (e.g., covalently or otherwise) to a first end of the second polynucleotide and the blocking analyte is linked to other end of the second polynucleotide and the redox reporter is linked to the antibody. In this configuration, the redox reporter is proximal to the sensing electrode when the antibody binds the blocking analyte, triggering a detectable signal from the electrode. When the antibody binds to the target analyte, the redox reporter is moved away from the sensing electrode, thereby allowing for a detectable change in signal.

As shown in FIG. 9, in some embodiments, a first polynucleotide strand links the antibody the redox reporter is linked to the antibody. A second polynucleotide strand that hybridizes to the first polynucleotide strand is linked to the blocking analyte such that the antibody can bind to the blocking analyte. The antibody can be tethered (linked) to the sensing electrode by a separate linker that can be a polynucleotide or non-polynucleotide (e.g., a bi-functional) linker. The first or second polynucleotide strand, or both, can be covalently or non-covalently linked to the antibody. In the absence of a target analyte, the antibody binds to the blocking analyte, which in view of hybridization between the two polynucleotide strands results in pulling the redox reporter away from the sensing electrode. However, when the target analyte displaces the blocking analyte the first and second polynucleotide strand will become closer to the sensing electrode, resulting in a detectable change of signal.

In certain embodiments, the sensing electrode can be a gold electrode. The linker can be engineered to contain a thiol functional group, which can form a gold-thiol bond between the linker and the gold electrode. Examples of redox reporters include, but are not limited to, methylene blue, thionine, anthraquinone, nile blue, gallocyanine, ferrocene, and pentamethyl ferrocene

In any of the single-antibody constructs described herein, the linker(s) can be a double-stranded polynucleotide or a single-stranded polynucleotide. In some embodiments, the linker can be a partially double-stranded polynucleotide comprising at least one unpaired nucleotide (e.g., between 1 and 20, between 1 and 18, between 1 and 16, between 1 and 14, between 1 and 12, between 1 and 10, between 1 and 8, between 1 and 6, between 1 and 4, between 1 and 3, between 2 and 20, between 4 and 20, between 6 and 20, between 8 and 20, between 10 and 20, between 12 and 20, between 14 and 20, between 16 and 20, or between 18 and 20 unpaired nucleotides; e.g., 1, 2, 3, 4, 5, 6, 7, 8, 9, 10, 11, 12, 13, 14, 15, 16, 17, 18, 19, or 20 unpaired nucleotides). In some embodiments, the linker can be a partially double-stranded polynucleotide, in which at least 1% (e.g., between 1% and 20%, between 1% and 18%, between 1% and 16%, between 1% and 14%, between 1% and 12%, between 1% and 10%, between 1% and 8%, between 1% and 6%, between 1% and 4%, between 2% and 20%, between 4% and 20%, between 6% and 20%, between 8% and 20%, between 10% and 20%, between 12% and 20%, between 14% and 20%, between 16% and 20%, or between 18% and 20%) of the length of the linker comprises unpaired nucleotide(s). The length of the linker can be at least 10 nucleotides, e.g., between 10 and 100 nucleotides (e.g., between 10 and 90, between 10 and 80, between 10 and 70, between 10 and 60, between 10 and 50, between 10 and 40, between 10 and 30, between 10 and 20, between 10 and 15 nucleotides, between 20 and 100, between 30 and 100, between 40 and 100, between 40 and 80, between 50 and 80, between 50 and 100, between 50 and 70, between 60 and 100, between 70 and 100, between and 100, or between 90 and 100; e.g., 10, 15, 20, 25, 30, 35, 40, 45, 50, 55, 60, 65, 70, 75, 85, 90, 95, or 100 nucleotides).

In certain embodiments, the unpaired nucleotides can be located at the 5′ terminus and/or the 3′ terminus of the linker or in the middle of the linker sequence. Further, the linker can contain one or more mismatched nucleotides (e.g., between 1 and 20, between 1 and 18, between 1 and 16, between 1 and 14, between 1 and 12, between 1 and 10, between 1 and 8, between 1 and 6, between 1 and 4, between 1 and 3, between 2 and 20, between 4 and 20, between 6 and 20, between 8 and 20, between 10 and 20, between 12 and 20, between 14 and 20, between 16 and 20, or between 18 and 20) mismatched nucleotides; e.g., 1, 2, 3, 4, 5, 6, 7, 8, 9, 10, 11, 12, 13, 14, 15, 16, 17, 18, 19, or 20 mismatched nucleotides). The linker can also or alternatively contain one or more bulges, which are unpaired stretches of nucleotides located within one strand of a double-stranded or partially double-stranded polynucleotide. Bulge sizes can vary from a single unpaired nucleotide up to several nucleotides (e.g., between 2 and 20, between 2 and 18, between 2 and 16, between 2 and 14, between 2 and 12, between 2 and 10, between 2 and 8, between 2 and 6, between 2 and 4, between 4 and 20, between 6 and 20, between 8 and 20, between 10 and 20, between 12 and 20, between 14 and 20, between 16 and 20, or between 18 and 20 nucleotides). Moreover, a linker in an antibody construct described herein can also contain one or more nicks (e.g., at least 1, 2, 3, 4, 5, 6, 7, 8, 9, or 10 nicks) in one or both strands of a double-stranded or partially double-stranded polynucleotide linker. A linker described herein can also contain one or more non-natural nucleotides (e.g., at least 1, 2, 3, 4, 5, 6, 7, 8, 9, or 10 non-natural nucleotides). Examples of non-natural nucleotides are described in, e.g., Stovall et al., Curr Protoc Nucleic Acid Chem 56:9.6.1-33, 2014; and Saito-Tarashima and Minakawa, Review Chem Pharm Bull (Tokyo), 66(2):132-138, 2018, and are also described in detail further herein. The presence of one or more mismatched nucleotides, bulges, nicks, non-natural nucleotides, and/or unpaired nucleotides can alter the flexibility and/or temporal sensitivity of the construct.

In a single-antibody construct described herein, in some embodiments, the blocking analyte and the target analyte can be the same. In some embodiments, the blocking analyte and the target analyte can be the different. In other embodiments, the blocking analyte can be a structural analog of the target analyte, in which one or more atoms or functional groups in the blocking analyte are different from the target analyte. For example, desthiobiotin is a structural analog of biotin and can be used as a blocking analyte in a single-antibody construct. In certain embodiments, a binding affinity of the antibody to the target analyte is between 90% and 110% (e.g., between 90% and 105%, between 90% and 100%, between 90% and 95%, between 95% and 110%, between 100% and 110%, or between 105% and 110%) of a binding affinity of the antibody to the blocking analyte. In certain embodiments, the antibody has a higher binding affinity for the target analyte than for the blocking analyte. In other embodiments, the antibody has a lower binding affinity for the target analyte than for the blocking analyte.

In some embodiments, a blocking analyte can be a molecule that comprises the antibody's cognate antigen, an epitope of the antibody's cognate antibody, or a fragment thereof. In some embodiments, a blocking analyte can be a molecule that can bind to the antibody in the antibody construct independent of the antibody's cognate antigen type. For example, a blocking analyte can be a protein that generally blocks antibody-antigen binding, for example, but not limited to, by binding to the antibody's conserved regions. A blocking analyte that binds to the antibody in the antibody construct independent of the antibody's cognate antigen type can be, e.g., Protein M (see, e.g., Grover et al., Science, 343(6171):656-661, 2014), Protein A, Protein G, and Protein L. In yet other embodiments, a blocking analyte that binds to the antibody in the antibody construct independent of the antibody's cognate antigen type can be a DNA aptamer or a nanostructure.

The antibody can be covalently conjugated to the linker using available conjugation techniques in the art (see, e.g., Gong et al., Bioconjugate Chem. 27(1):217-225, 2016). For example, the antibody can be covalently conjugated to the linker via its Fc region.

### Dual-Antibody Construct

An antibody construct disclosed herein can be a dual-antibody construct. In one example, a dual-antibody construct can comprise: (a) two detecting strands, wherein a first detecting strand comprises a first antibody or a binding fragment thereof, and a second detecting strand comprises a second antibody or a binding fragment thereof; and (b) a first label and a second label, in which the detecting strands or portions thereof are complementary and hybridize to each other or one or more scaffold strands, and wherein in the presence of the target analyte, the first antibody and the second antibody binds to two different epitopes on the target analyte, and the first and second labels interact with each other to generate a detectable readout compared to when there is an absence of the target analyte.

In some embodiments of the dual-antibody construct, the first label is linked to the first antibody and the second label is linked to the second antibody. In other embodiments, the first label and/or the second label can be linked to one or more scaffold strands. For example, the first label and the second label can each be linked to a separate scaffold strand. In another example, the first label can be linked to one terminus of the scaffold strand and the second label can be linked to the other terminus of the same scaffold strand. In the presence of a target analyte, the first antibody and the second antibody can bind to different epitopes on the target analyte, bringing the first label and the second label in proximity of each other to generate a detectable readout.

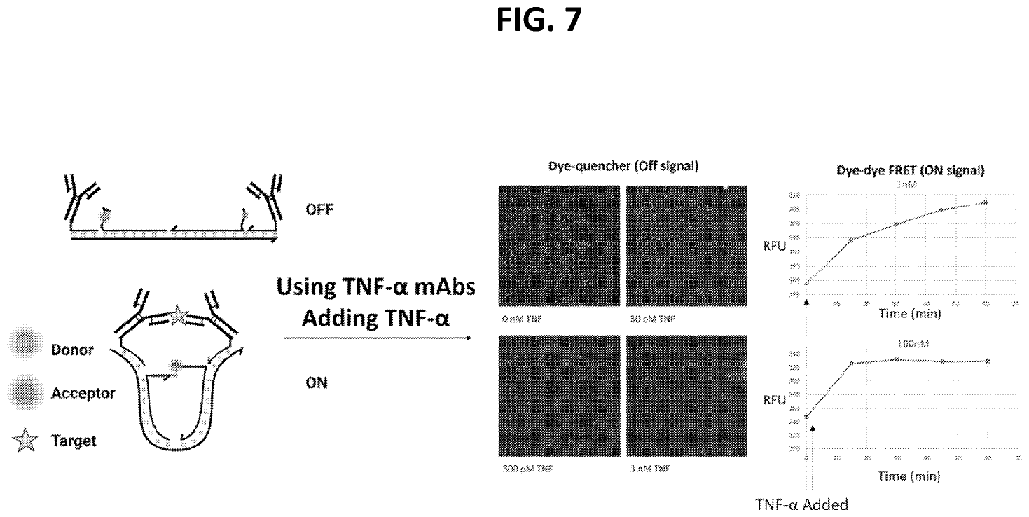

In one example of a dual-antibody construct, the antibody construct can contain a scaffold strand and two detecting strands, wherein each of the two detecting strands or a portion thereof is complementary and hybridizes to the first scaffold strand or a portion thereof (see, e.g., FIG. 7). In some embodiments, the scaffold strand can be at least 100 nucleotides long, e.g., between 100 and 200 (e.g., between 100 and 180, between 100 and 160, between 100 and 140, between 100 and 120, between 120 and 200, between 140 and 200, between 160 and 200, or between 180 and 200) nucleotides long. In specific embodiments, the first scaffold strand can be between 150 and 200 (e.g., between 150 and 190, between 150 and 180, between 150 and 170, between 150 and 160, between 160 and 200, between 170 and 200, between 180 and 200, or between 190 and 200) nucleotides long; the second scaffold strand can be between 90 and 140 (e.g., between 90 and 130, between 90 and 120, between 90 and 110, between 90 and 100, between 100 and 140, between 110 and 140, between 120 and 140, or between 130 and 140) nucleotides long; and optionally each of the detecting strands can be 10-50, e.g., 20-40 or 25-35 nucleotides long. The length of each of the scaffold strands and detecting strands can be modified independently.

Further, in some embodiments of the dual-antibody construct, the detecting strands and/or the scaffold strand(s) can comprise one or more mismatched nucleotides. The presence of one or more mismatched nucleotides can alter the flexibility and/or temporal sensitivity of the construct.

In some embodiments of the dual antibody aspect, one or both antibodies are linked directly to the labels (e.g., the first label is linked to the first antibody and the second label is linked to the second antibody). In these aspects, binding of the antibodies to a target analyte bring the first and second labels in proximity allowing for a change in detectable signal compared to in the absence of the target analyte.

In some embodiments, the first label and/or second label is linked to a polynucleotide strand (e.g., a “label oligonucleotide”) that hybridizes to a portion of the scaffold. See, e.g., FIG. 7. In these embodiments, while the labels are not covalently linked to the antibodies, the proximity of the first and second label to each other are dictated in part by whether the antibodies bind to the same target analyte or not. See, FIG. 7. In some embodiments, when the antibodies bind to the target analyte, the label oligonucleotides linked to the labels hybridize (e.g., 3′ regions or 5′ regions of the label oligonucleotide hybridize). The hybridizing regions can be relatively short, e.g., 1, 2, 3, 4, 5, 6, 7, or more nucleotides, to stabilize the labels together but sufficiently weakly hybridized such that in the absence of the target analyte the regions do not hybridize. In other embodiments, the regions of the label oligonucleotide are not complementary but the labels are nevertheless brought into proximity by the binding of the antibodies. See, e.g., FIG. 7.

In some embodiments, the first label and/or second label is linked to a polynucleotide strand that is part of the scaffold. In these embodiments, the labels are brought into proximity by the binding of the antibodies.

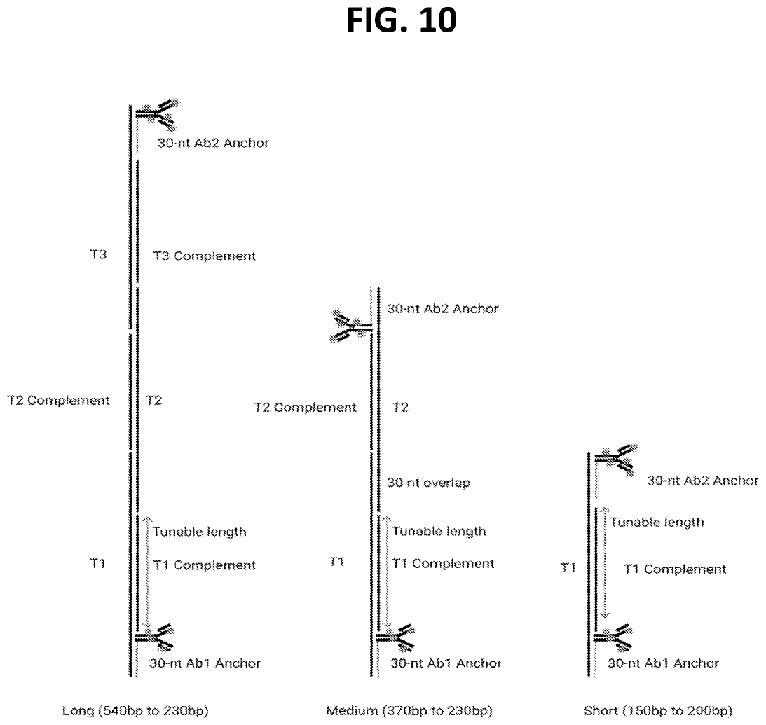

The first and second antibodies are each linked to a detecting polynucleotide strand. These detecting strands can have complementary ends allowing for them to directly hybridize. Alternatively, the polynucleotide strands can hybridize to one or more scaffold strands to indirectly link the two antibodies. For example, FIG. 10 depicts aspects in which scaffold polynucleotides hybridize to each other and then the antibody polynucleotides hybridize at either end. FIG. 10 also depicts optional lengths of the scaffolds, allowing for regulation of the activity and signal generation of the dual antibody constructs.

### VI. Labels

An antibody construct described herein comprises labels that function to produce a detectable readout when the antibody construct is in a reporting state, i.e., when a target analyte is present. In some embodiments, the labels can produce a chemical and/or physical signal as a detectable readout when the antibody construct binds to a target analyte.

In some embodiments, the antibody construct generates fluorescence as a detectable readout when it binds to a target analyte. In some embodiments, one of the first and second labels on the antibody construct can be a fluorophore and the other of the first and second labels can be a quencher. In this case, the blocking analyte binds to the antibody in the absence of the target analyte and the fluorescence signal is quenched.

In some embodiments, one of the first and second labels on the antibody construct can be a donor fluorophore and the other of the first and second labels can be an acceptor fluorophore and the first and second labels can form a FRET pair. It is known that, in order for two fluorophores to be FRET partners, the emission spectrum of the donor fluorophore must partially overlap the excitation spectrum of the acceptor fluorophore. In some embodiments, the preferred FRET-partner pairs are those for which the value R0 (Förster distance, distance at which energy transfer is 50% efficient) is greater than or equal to 30Å. Examples of FRET partners are known in the art, see, e.g., Massey et al., Analytica Chimica Acta 568:181-189, 2006. In some embodiments, when the antibody is bound by the blocking analyte in the absence of a target analyte, the first and second labels are in proximity of each other to produce a FRET signal. The disappearance or reduction of the FRET fluorescence can serve as a signal of target analyte binding. In other words, in the absence of the target analyte, the fluorescent signal from the acceptor fluorophore can serve as the detectable signal. In the presence of the target analyte and the formation of the antibody-target analyte complex, the donor fluorophore and the acceptor fluorophore are not in proximity of each other to produce a FRET signal and the fluorescent signal of the donor fluorophore can serve as the detectable readout. Other fluorescence based methods that can be used to investigate the structure, binding, and dynamics of an antibody construct can be found in, e.g., Perez-Gonzales et al., Front Chem. 4:33, 2016.

Examples of fluorophores, as well as quenchers, are known in the art, e.g., as described in Marras, Methods Mol Biol. 335:3-16, 2006; Kozma and Kele, Org Biomol Chem. 17(2):215-233, 2019; and Wang et al., Angew Chem Int Ed Engl. Mar. 7, 2019. Efficient and complete quenching of the fluorescence emitted from the fluorophore by the quencher depends in part on the overlap between the fluorophore emission and quencher absorption spectra. For example, fluorophore coumarin emits at emission wavelength around 472 nm and can be paired with quencher QSY35 which absorbs at wavelength around 475 nm. In another example, fluorophore Alexa 532 emits at emission wavelength around 554 nm and can be a paired with quencher QSY7 which absorbs at wavelength around 560 nm. In yet another example, fluorophore Alexa 647 emits at emission wavelength around 665 nm and can be paired with quencher QSY21 which absorbs at wavelength around 661 nm.

In other embodiments, a label can be a fluorophore whose fluorescence can be quenched. An example of such a fluorophore is 2-amino purine, whose fluorescence can be quenched when it is stacked with purines and/or pyrimidines (see, e.g., Jean and Hall, Proc Natl Acad Sci USA. 98(1):37-41, 2001).

In other embodiments, the labels in the antibody construct can produce chemical and/or physical signals as a detectable readout when the antibody construct binds to a target analyte. These signals can be monitored to infer binding to the target analyte. In one example, the labels can be electrochemical reporters (see, e.g., Ferguson et al., Sci Transl Med. 5(213):213ra165, 2013). A first label can be an electrode (e.g., a gold electrode) and a second label can be a redox reporter (e.g., methylene blue). In some embodiments, upon binding to the target analyte, the antibody construct undergoes a conformational rearrangement that modulates the redox current and generates an electrochemical signal.

### V. Non-Natural Nucleotides

In some embodiments, the antibody constructs can include one or more non-natural nucleotides (e.g., non-natural DNAs and/or non-natural RNAs). A non-natural nucleotide can include one or more of a non-natural nucleobase, a non-natural sugar, and a non-natural internucleoside linkage.

### Non-Natural Nucleobases

A non-natural nucleobase refers to a nucleobase having at least one change that is structurally distinguishable from a naturally-occurring nucleobase (i.e., adenine, guanine, cytosine, thymine, or uracil). In some embodiments, a non-natural nucleobase is functionally interchangeable with its naturally-occurring counterpart. Both naturally-occurring and non-natural nucleobases are capable of hydrogen bonding. Modifications on non-natural nucleobases may help to improve the stability of the antibody constructs to nucleases. In some embodiments, an antibody construct described herein may include at least one non-natural nucleobase. Examples of non-natural nucleobases include, but are not limited to, 5-methylcytosine, 5-hydroxymethyl cytosine, xanthine, hypoxanthine, 2-aminoadenine, 6-methyladenine, 6-methylguanine, 2-propyladenine, 2-propylguanine, 2-thiouracil, 2-thiothymine, 2-thiocytosine, 5-halouracil, 5-halocytosine, 5-propynyluracil, 5-propynylcytosine, 6-azouracil, 6-azocytosine, 6-azothymine, 5-uracil (pseudouracil), 4-thiouracil, 8-haloadenine, 8-aminoadenine, 8-thioladenine, 8-thioalkyladenine, 8-hydroxyladenine, 8-haloguanine, 8-aminoguanine, 8-thiolguanine, 8-thioalkylguanine, 8-hydroxylguanine, 5-halouracil, 5-bromouracil, 5-trifluoromethyluracil, 5-halocytosine, 5-bromocytosine, 5-trifluoromethylcytosine, 7-methylguanine, 7-methyladenine, 2-fluoroadenine, 2-aminoadenine, 8-azaguanine, 8-azaadenine, 7-deazaguanine, 7-deazaadenine, 3-deazaguanine, and 3-deazaadenine. In some embodiments, an antibody construct described herein has one or more non-natural nucleobases (e.g., 5-methylcytosine).

### Non-Natural Sugars

A non-natural sugar refers to a sugar having at least one change that is structurally distinguishable from a naturally-occurring sugar (i.e., 2′-deoxyribose in DNA or ribose in RNA). Modifications on non-natural sugars may help to improve the stability of the antibody constructs to nucleases. In some embodiments, the sugar is a pentofuranosyl sugar. The pentofuranosyl sugar ring of a nucleoside may be non-natural in various ways including, but not limited to, addition of a substituent group, particularly, at the 2′ position of the ring; bridging two non-geminal ring atoms to form a bicyclic sugar (i.e., a locked sugar); and substitution of an atom or group such as —S—, —N(R)— or —C(R₁)(R₂) for the ring oxygen. Examples of non-natural sugars include, but are not limited to, substituted sugars, especially 2′-substituted sugars having a 2′—F, 2′—OCH₂ (2′—OMe), or a 2′—O(CH₂)₂—OCH₃ (2′—O—methoxyethyl or 2′—MOE) substituent group; and bicyclic sugars. A bicyclic sugar refers to a non-natural pentofuranosyl sugar containing two fused rings. For example, a bicyclic sugar may have the 2′ ring carbon of the pentofuranose linked to the 4′ ring carbon by way of one or more carbons (i.e., a methylene) and/or heteroatoms (i.e., sulfur, oxygen, or nitrogen). The second ring in the sugar limits the flexibility of the sugar ring and thus, constrains the polynucleotide in a conformation that is favorable for base pairing interactions with its target nucleic acids. An example of a bicyclic sugar is a locked sugar, which is a pentofuranosyl sugar having the 2′-oxygen linked to the 4′ ring carbon by way of a carbon (i.e., a methylene) or a heteroatom (i.e., sulfur, oxygen, or nitrogen). In some embodiments, a locked sugar has the 2′-oxygen linked to the 4′ ring carbon by way of a carbon (i.e., a methylene). In other words, a locked sugar has a 4′—(CH₂)—O—2′ bridge, such as α-L-methyleneoxy (4′—CH₂—O—2′) and β-D-methyleneoxy (4′—CH₂—O—2′). A nucleoside having a lock sugar is referred to as a locked nucleoside.

Other examples of bicyclic sugars include, but are not limited to, (6′S)-6′ methyl bicyclic sugar, aminooxy (4′—CH₂—O—N(R)—2′) bicyclic sugar, oxyamino (4′—CH₂—N(R)—O—2′) bicyclic sugar, wherein R is, independently, H, a protecting group or C1-C12 alkyl. The substituent at the 2′ position can also be selected from allyl, amino, azido, thio, O-allyl, O—C1-C10 alkyl, OCF₃, O(CH₂)₂SCH₃, O(CH₂)₂—O—N(Rₘ)(Rₙ), and O—CH₂—C(═O)—N(Rₘ)(Rₙ), wherein each Rₘ and Rₙ is, independently, H or substituted or unsubstituted C1-C10 alkyl.

In some embodiments, a non-natural sugar is an unlocked sugar. An unlocked sugar refers to an acyclic sugar that has a 2′, 3′-seco acyclic structure, where the bond between the 2′ carbon and the 3′ carbon in a pentofuranosyl ring is absent.

### Non-Natural Internucleoside Linkages

An internucleoside linkage refers to the backbone linkage that connects the nucleosides. An internucleoside linkage may be a naturally-occurring internucleoside linkage (i.e., a phosphate linkage, also referred to as a 3′ to 5′ phosphodiester linkage, which is found in DNA and RNA) or a non-natural internucleoside linkage. A non-natural internucleoside linkage refers to an internucleoside linkage having at least one change that is structurally distinguishable from a naturally-occurring internucleoside linkage. Non-natural internucleoside linkages may help to improve the stability of the antibody constructs to nucleases and enhance cellular uptake.

Examples of non-natural internucleoside linkages include, but are not limited to, a phosphorothioate linkage, a phosphorodithioate linkage, a phosphoramidate linkage, a phosphorodiamidate linkage, a thiophosphoramidate linkage, a thiophosphorodiamidate linkage, a phosphoramidate morpholino linkage, and a thiophosphoramidate morpholino linkage, and a thiophosphorodiamidate morpholino linkage, which are known in the art and described in, e.g., Bennett and Swayze, Annu Rev Pharmacol Toxicol. 50:259-293, 2010. A phosphorothioate linkage is a 3′ to 5′ phosphodiester linkage that has a sulfur atom for a non-bridging oxygen in the phosphate backbone of a polynucleotide. A phosphorodithioate linkage is a 3′ to 5′ phosphodiester linkage that has two sulfur atoms for non-bridging oxygens in the phosphate backbone of a polynucleotide. A thiophosphoramidate linkage refers to a 3′ to 5′ phospho-linkage that has a sulfur atom for a non-bridging oxygen and a NH group as the 3′-bridging oxygen in the phosphate backbone of a polynucleotide. In some embodiments, an antibody construct described herein has at least one phosphorothioate linkage. In some embodiments, all of the internucleoside linkages in an antibody construct described herein are phosphorothioate linkages.

### VI. Methods

The disclosure also provides methods of detecting a target analyte in a sample (e.g., a biological sample), comprising: (1) contacting the sample with an antibody construct described herein; and (2) measuring binding of the antibody construct to the target analyte using a detectable readout (e.g., an optical signal, an electrical signal, an electrochemical signal, a nuclear magnetic resonance signal, or a biological signal) from the antibody construct.

Further, the disclosure also includes methods of adjusting kinetics and/or effective binding affinity of an antibody construct, comprising: (1) generating an antibody construct described herein; (2) measuring binding of the antibody construct to a target analyte; (3) changing one or more components of the antibody construct; (4) re-measure binding of the antibody construct to the target analyte; and (5) optionally repeat steps (3) and (4) until the desired kinetics and/or effective binding affinity of the antibody construct is reached. One component that can be tailored to adjust the kinetics and/or effective binding affinity of the antibody construct is the length and/or nucleotide composition of the linker. In some embodiments, increasing or decreasing the length of the linker can provide or remove flexibility and/or sensitivity of the antibody construct. Further, introducing or removing one or more mismatched nucleotides, bulges, nicks, non-natural nucleotides, and/or unpaired nucleotides from the linker can also provide or remove flexibility and/or sensitivity of the antibody construct. Another component that can be tailored to adjust the kinetics and/or effective binding affinity of the antibody construct is the identity of the blocking analyte. In some embodiments, changing the identity of the blocking analyte can vary the affinity of the antibody for the blocking analyte and thus the strength of intramolecular binding competition, or can vary the dissociation and association rates of the blocking analyte and thus the rate of target binding and blocking.

In a dual-antibody construct, increasing or decreasing the length of one or more scaffold strands provide or remove flexibility and/or sensitivity of the antibody construct. Furthermore, introducing or removing one or more mismatched nucleotides, bulges, nicks, non-natural nucleotides, and/or unpaired nucleotides from one or more scaffold strands can also provide or remove flexibility and/or sensitivity of the antibody construct.

The reversible, modular, and programmable nature of the antibody constructs enables the kinetics and the effective binding affinity of the antibody construct to be fine-tuned for the desired purpose and use of the construct, such as applications in molecular diagnostics, biomedical imaging, point-of-care devices, and home-use tests. Depending on the use format, the antibody construct can be modulated to have rapid, quantitative, sensitive, and multiplexed target analyte detection in complex systems.

### EXAMPLES

The antibody switch construct depicted in FIG. 1 was immobilized on protein G functionalized magnetic beads through interaction of the antibody Fc region by incubating the beads with 250 nM antibody switch construct in 1× PBS for 30 minutes. Free antibody was removed by washing the beads in 1× PBS, then the antibody switch-coated beads were incubated in 1× PBS spiked with varying concentrations of free biotin for 30 minutes. The fluorescent response of the switches was measured using either a spectrophotometer or flow cytometer to measure the Cy5 emission spectrum.

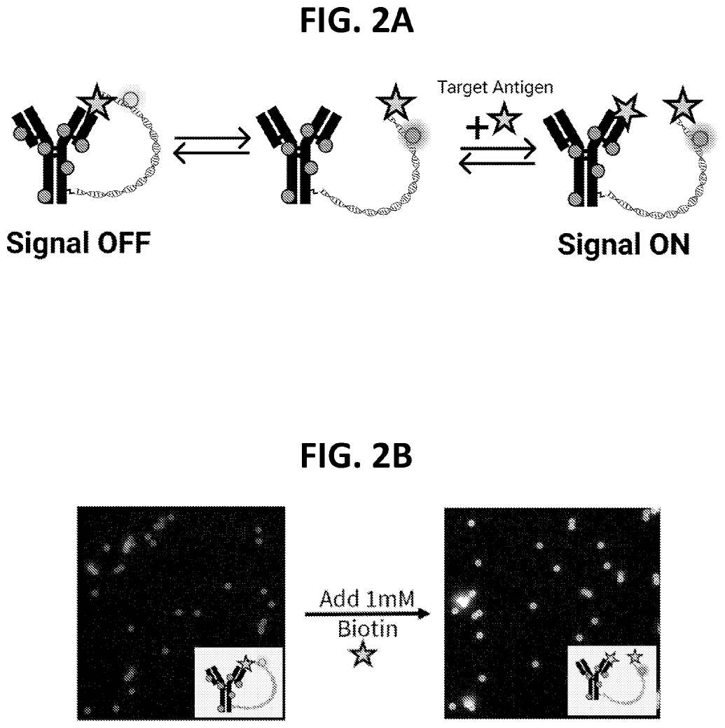

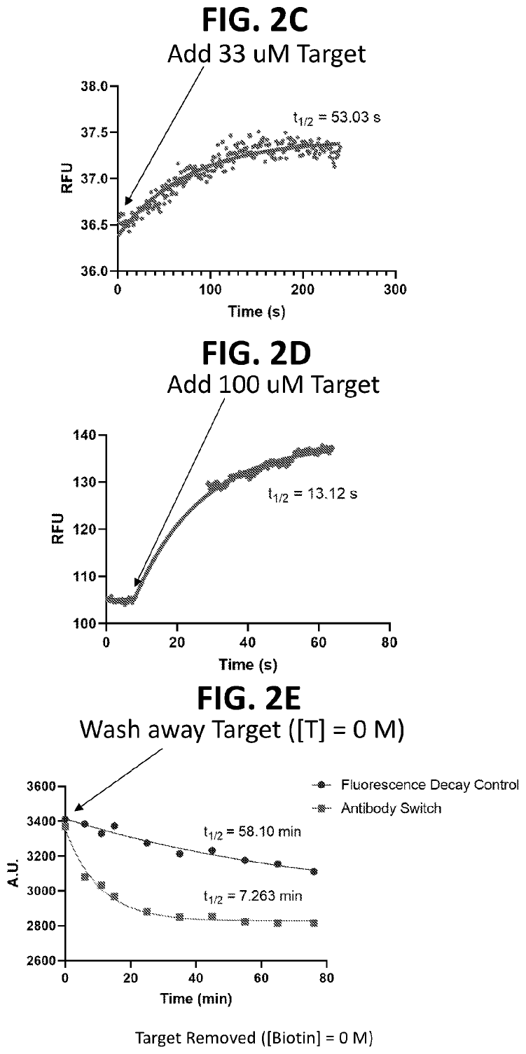

Antibody switch constructs depicted in FIG. 2 were immobilized on beads following the protocol described for FIGS. 1A-1D. Silanized glass coverslips were prepared using chemical vapor deposition of dimethyldichlorosilane and adhesive fluidics chambers were fixed overtop the coverslips. Antibody switch-coated beads were adhered to the coverslip surface through hydrophobic interactions by incubating the bead solution in 1× PBS within the chamber for 15 minutes. Excess beads were washed away by flowing 1× PBS through the chamber, then the surface was further passivated by incubating the chamber with 1% TWEEN-20 in 1× PBS for 10 minutes and washing with 1× PBS for 10 minutes. Kinetic responses of the antibody switches were then measured using fluorescent microscopy to observe switching-induced changes in Cy5 fluorescence. Various concentrations of biotin spiked into 1× PBS were flowed through the chamber as Cy5 fluorescence images were obtained at regular time intervals to observe the time dependence of switching.

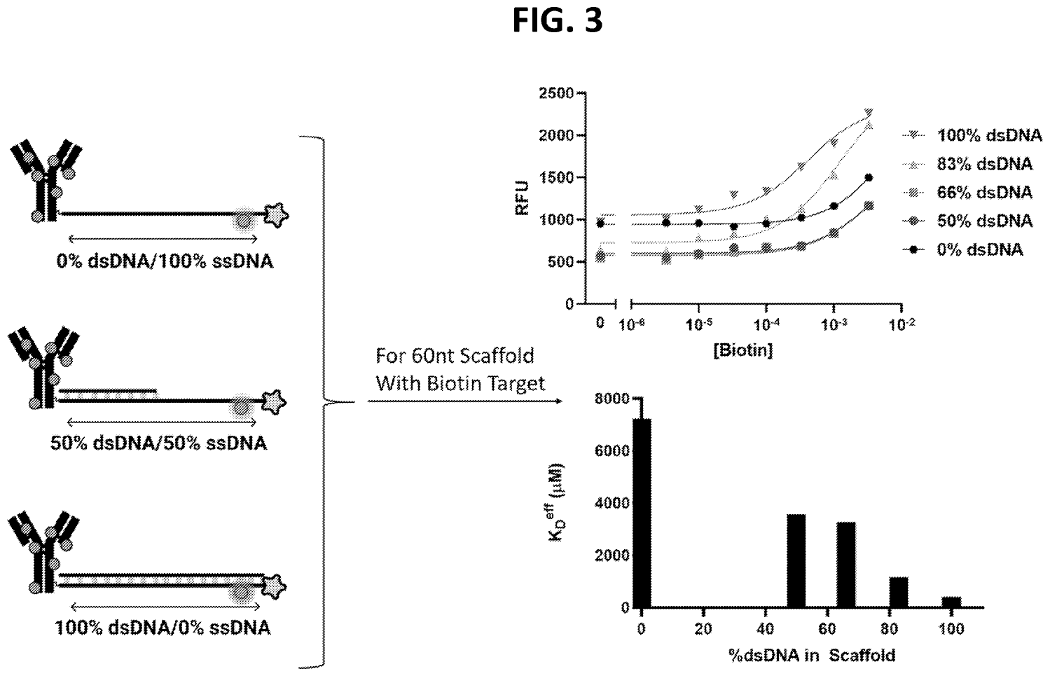

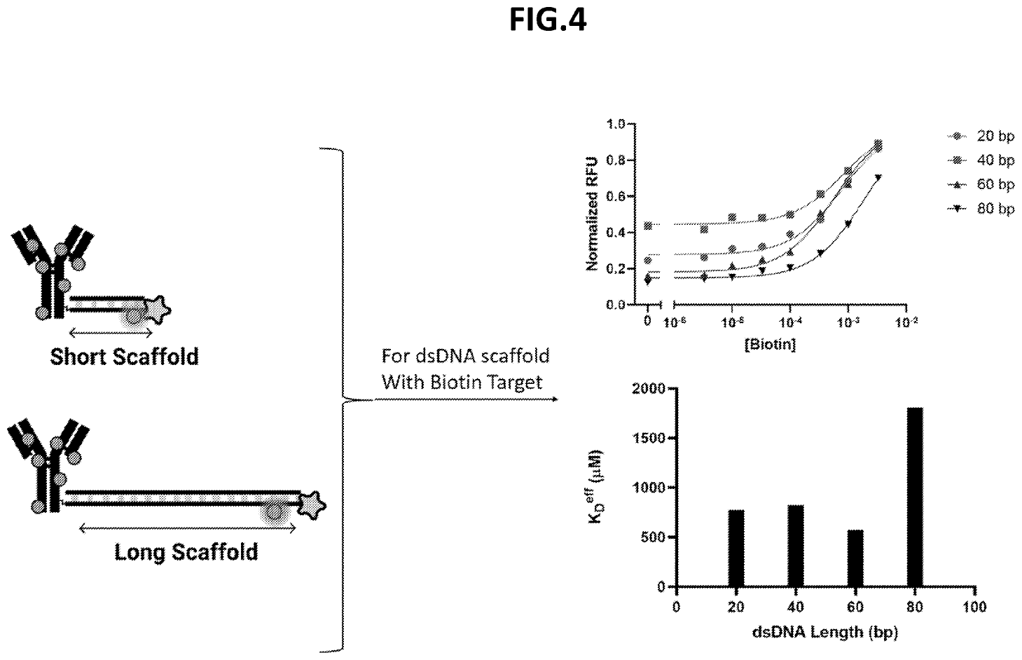

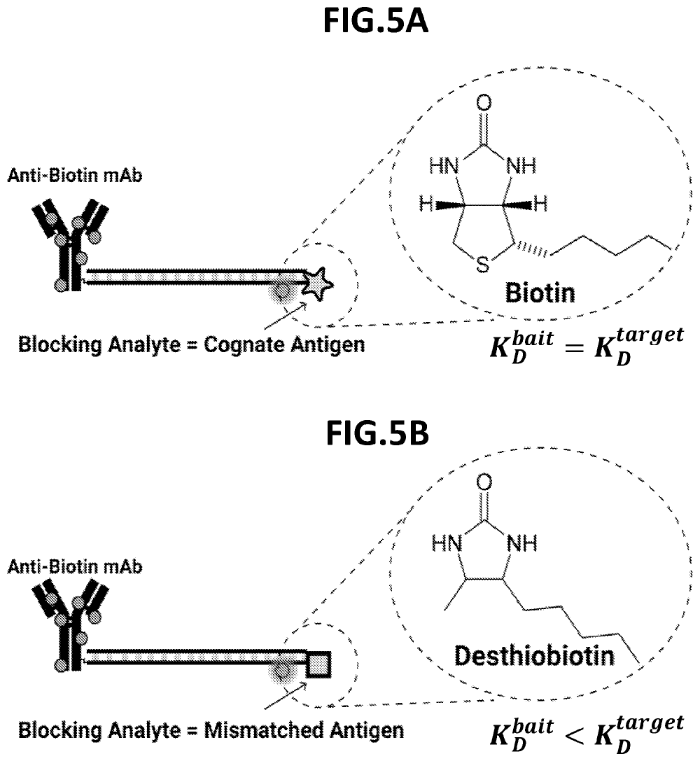

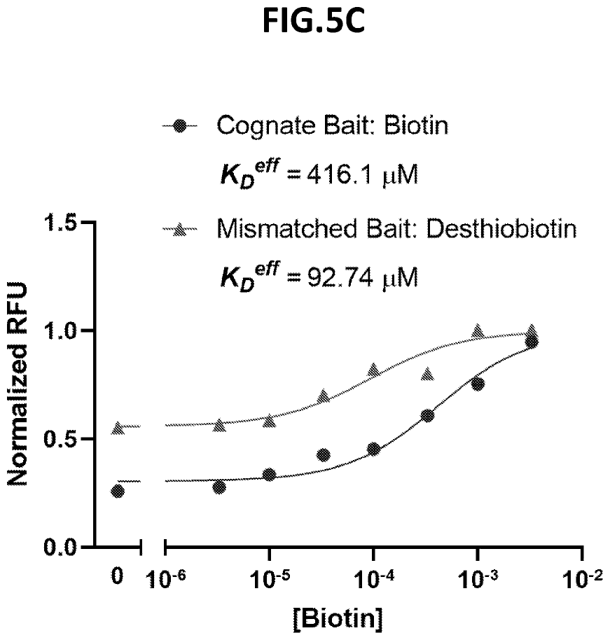

Antibody switch constructs depicted in FIG. 3-5 were synthesized using a variety of scaffold and blocking analyte designs. These constructs were subsequently functionalized on beads and their target-dependent fluorescent responses were measured using flow cytometry as described for FIGS. 1A-1D, to allow us to investigate how design changes alter the construct's effective binding affinity. In some instances (FIG. 3), constructs were synthesized with a 60nt scaffold, hen incubated in 1× PBS with an excess of single stranded DNA of various lengths designed to be complementary to different portions of the scaffold strand. This allows control over the number of double stranded bases within the scaffold. In other instances (FIG. 4), constructs were synthesized with a variety of scaffold lengths between 20 and 80 bp then incubated in 1× PBS with an excess of single stranded DNA of various lengths designed to be fully complementary to the scaffold strand. This forms fully double-stranded scaffolds and allows us to study the impact of length dependence on affinity. In other instances (FIG. 5), constructs were synthesized with scaffolds linked to different blocking analytes with different affinities for the antibody. Biotin has an affinity equal to the target binding affinity for biotin-specific antibodies, while a molecular analogue desthiobiotin has a lower affinity. This allows us to study the impact of bait identity on switch affinity. In all these cases, the fluorescence data over a wide range of biotin concentrations was fit with a Langmuir isotherm model in order to determine shifts in the effective switch affinity.

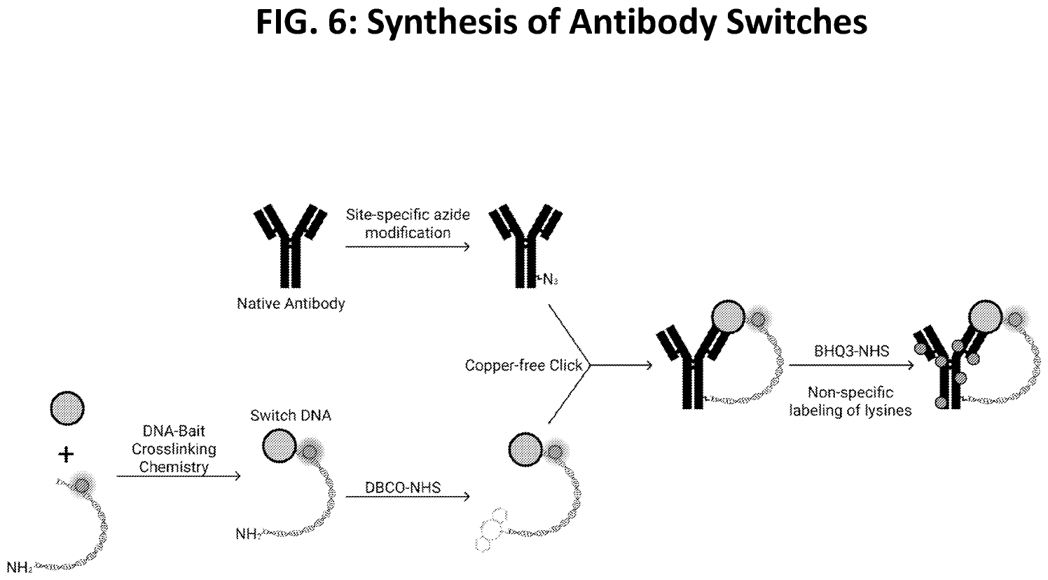

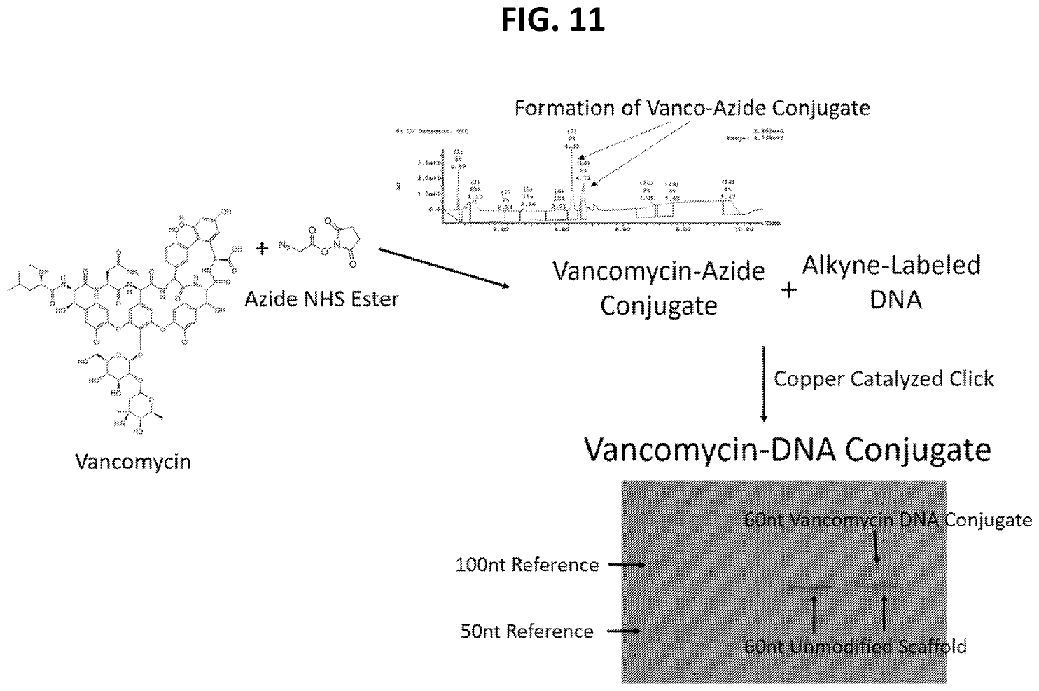

Antibody constructs depicted in FIG. 6 were synthesized through multi-step process. First, the blocking analyte is linked to the scaffold strand, either during solid-phase synthesis of the DNA scaffold, or through chemical crosslinking (see FIG. 11 for example of chemical crosslinking). The scaffold strand incorporates a label as shown in this example. The end of the scaffold strand not linked to the blocking analyte has an incorporated amino group which is functionalized with a DBCO moiety by incubating 5 uL of 15 mM DBCO NHS Ester in dimethylformamide to 56 uL of 90 uM DNA scaffold in 0.1 M sodium bicarbonate buffer for 4 hours at room temperature, followed by ethanol precipitation to remove free DBCO. The antibody is enzymatically modified to label an azide moiety at 1-4 glycosylation sites within the Fc region of the antibody (using the commercial Siteclick Antibody Azido Modification Kit, Thermo-Fisher). A copper-free click reaction between the azide and DBCO moiety is used to covalently link the scaffold and antibody, with the reaction done overnight at a 4:1 excess of DNA:Ab in Tris buffer at room temperature followed by purification with a 50 kDa MWCO size exclusion column. Finally, in some embodiments a second label is added onto the antibody strand, for example though conjugation of Quencher-NETS Esters to lysine residues throughout the antibody structure as shown in this example. Quencher-antibody conjugation is achieved by adding 1 uL of 1 mM BHQ3 NHS Ester to 75 uL of ˜4 uM antibody-scaffold construct in 0.1 M bicarbonate buffer, then incubating for 1 hour at room temperature. Final purification is performed using a 50 kDa MWCO size exclusion column.

The antibody switch construct depicted in FIG. 8 was synthesized with methylene blue labels on the antibody strand and a 60nt DNA scaffold linking the antibody to a biotin blocking analyte. A gold electrode surface was functionalized through gold-thiol bonding with 60nt single-stranded DNA complementary to the scaffold and passivated with mercaptohexanol. The electrode surface was incubated with the antibody construct in 1× PBS to allow hybridization between the surface-tethered DNA strand and the scaffold strand. This links the antibody construct to the surface and makes the scaffold strand assume double stranded form. Cyclic voltammetry scanning was used to measure changes in the redox current as the antibody-functionalized electrode surface was incubated in buffer spiked with various concentration of biotin. This allows the observation of target-dependent electrical signaling.

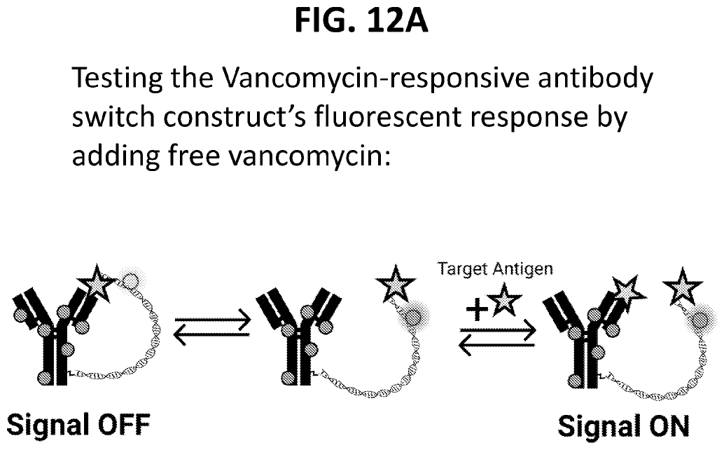

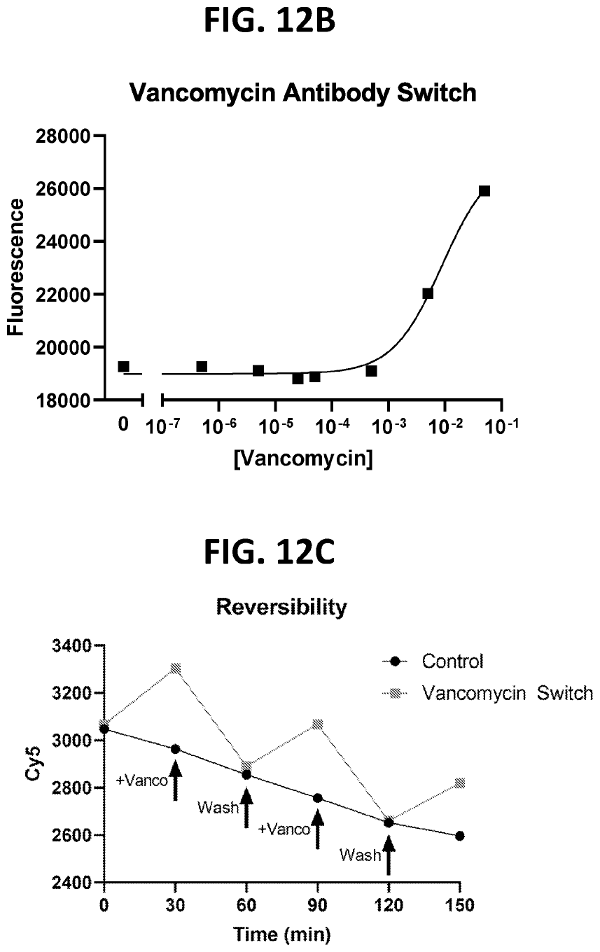

The following discussion and FIGS. 11-12 show the synthesis scheme for achieving DNA crosslinking of a vancomycin blocking analyte, and the resulting data for fluorescent switching and reversibility. Synthesis pathway for the vancomycin-DNA conjugate, where the DNA serves as the scaffold strand and linked vancomycin serves as the blocking analyte. See FIG. 11. Vancomycin reacts with an Azide NHS Ester through a primary amine in the structure. Formation of this conjugate is verified using liquid chromatography-mass spectrometry, which identifies a ˜50% yield of reacted vancomycin. The DNA scaffold is synthesized with a terminal alkyne moiety, allowing the vancomycin-azide conjugate to be linked to the scaffold through copper catalyzed click chemistry. Formation of the complete vancomycin-DNA conjugate product can be visualized on gel electrophoresis due to the mobility difference of vancomycin-DNA.

A fluorescently-reporting single antibody switch construct was synthesized from a vancomycin-specific monoclonal IgG and the vancomycin-DNA conjugate (60nt DNA) described above using the same process outlined in FIG. 6. The resulting construct was incubated with DNA complementary to the scaffold to form a double stranded scaffold, then was immobilized on beads. See FIG. 12. Changes in the antibody switch fluorescence in response to varying concentrations of vancomycin spiked into 1× PBS buffer were measured on flow cytometry (FIG. 12, bottom left). The results show significant vancomycin-dependent activation of fluorescence. Further, these switches were immobilized on beads and were repeatedly cycled through conditions of high vancomycin concentration and no vancomycin in buffer to study the reversibility of the switch (FIG. 12, bottom right). While we observe some slow decrease in the fluorescence baseline (identical between control and the vancomycin switch) likely due to photobleaching and antibody construct dissociation from beads, the switching mechanism is reversible and repeatable over multiple cycles of concentration increases and decreases. The switches settle to steady state fluorescence within 30 minutes of concentration changes, and the amplitude of the fluorescent response to the same vancomycin concentration is maintained over three cycles.

### Biotin Antibody Switch Reversibility

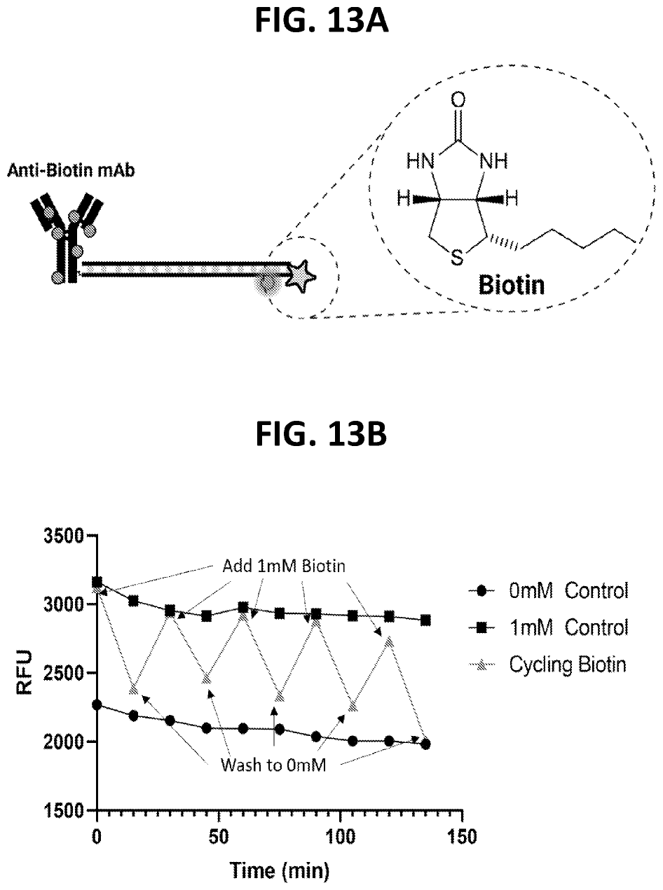

A surface-tethered bead-based reversibility assay showed that the biotin-responsive single antibody switch (FIG. 13A) can be used to continuously measure changing analyte concentrations through many cycles of increasing or decreasing concentrations. The fluorescent assay data (FIG. 13B) showed that as compared to control experiments where analyte concentration did not change (0 mM and 1 mM control), changing concentrations (alternating between 0 and 1 mM every 15 minutes) were rapidly reflected in the fluorescent signal from the switch. These changing signals were stable over time, which is critical to real-time continuous biosensing as an ideal sensor will be able to track analyte concentrations over long periods of time.

### Biotin Antibody Switch Dissociation Kinetics

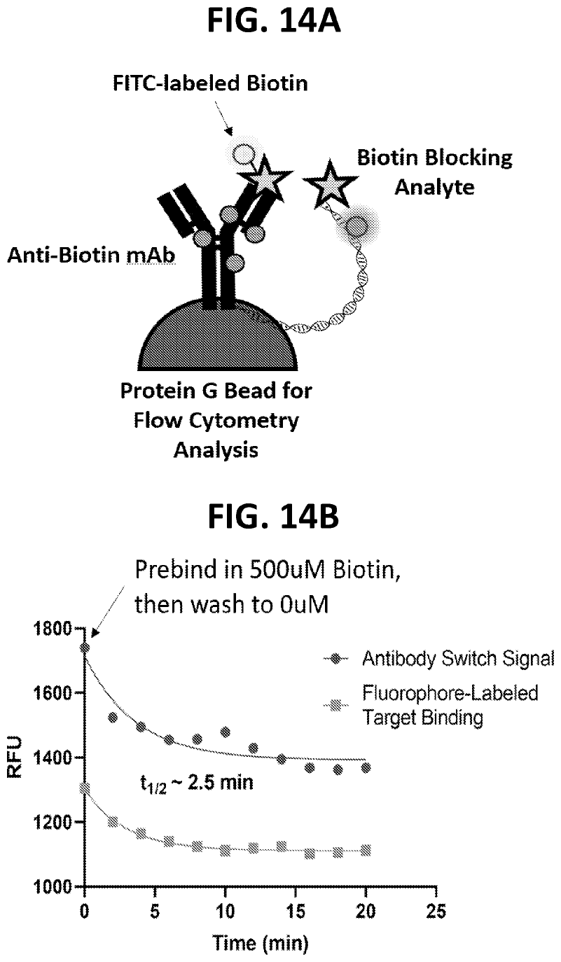

A surface-tethered bead-based assay showed that the biotin-responsive single antibody switch responded to changing target concentration with fast temporal resolution that enables rapid measurement. The antibody switch construct (FIG. 14A) was immobilized on a bead for flow cytometry and was prebound to a fluorescently labeled target analog (FITC-biotin). The target was then washed away, and flow cytometry was used to measure the temporal response (FIG. 14B) in signal from both the fluorescent target and intrinsic antibody switch signaling. These results were well correlated, showing that the antibody switch accurately reflected the underlying target concentrations. This experiment also showed that the antibody switch can respond with ˜2.5 min time resolution which can enable rapid measurements.

### Digoxigenin Responsive Antibody Switch

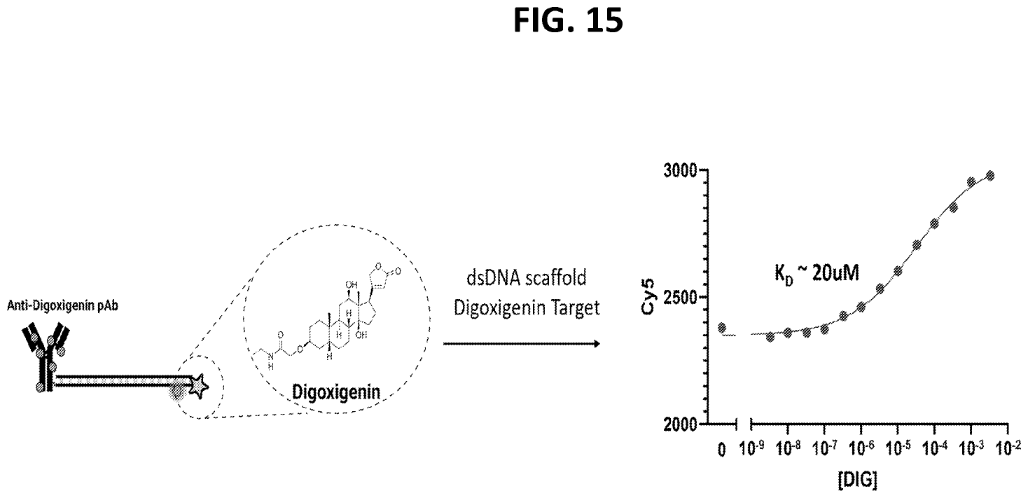

The antibody-switch construct employs a single antibody and achieves target-responsive molecular switching in response to a small molecule steroid, digoxigenin (DIG). In contrast to the previous results which all demonstrated biotin-responsive behavior, this construct expands the design to function in sensing a new molecule. The design leverages the same architecture as shown in previous results, but replaces the anti-biotin monoclonal antibody with an anti-digoxigenin polyclonal antibody. The blocking strand, biotin in the previous embodiment, was replaced with digoxigenin. Surface-tethered bead-based measurements of concentration-dependent switching (FIG. 15) showed the fluorescence was concentration-dependent in response to DIG.

### Digoxigenin Switch Reversibility

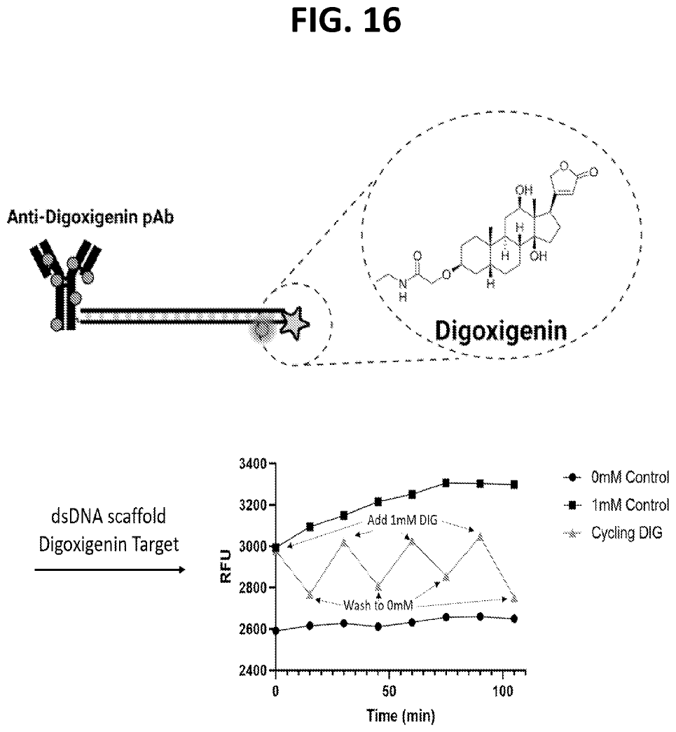

A surface-tethered bead-based reversibility assay showed that the digoxigenin-responsive single antibody switch can be used to continuously measure changing analyte concentrations through changing fluorescent output over many cycles of increasing or decreasing concentrations (FIG. 16).

### Digoxigenin Switch Synthesis

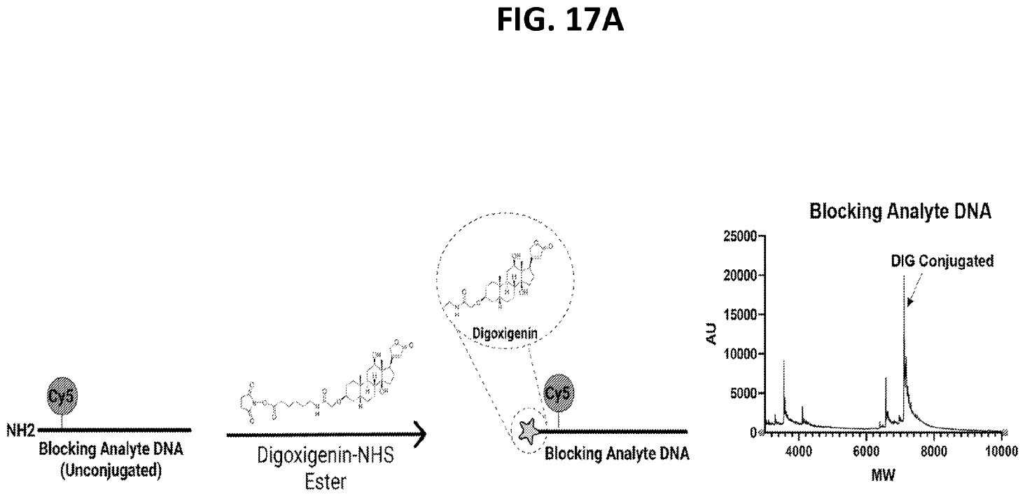

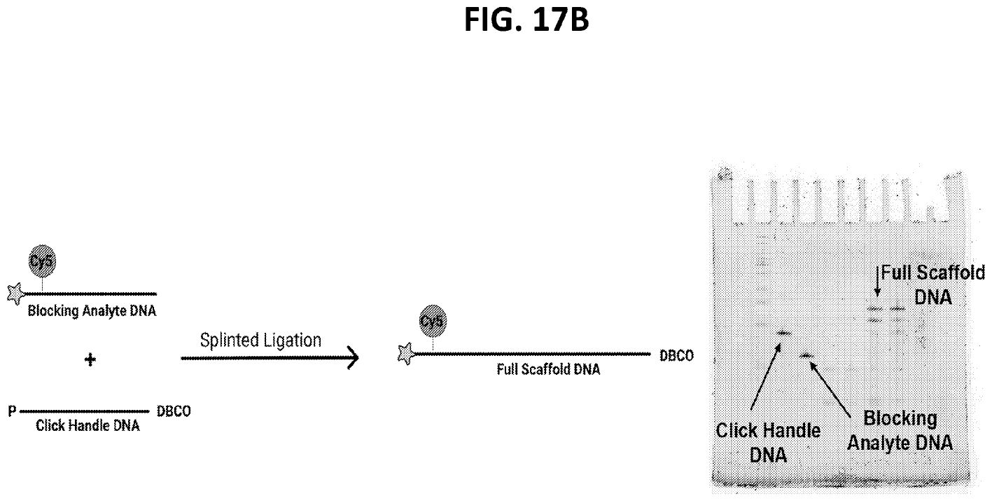

FIGS. 17A and 17B show the general synthesis workflow for synthesizing the scaffold and blocking strand for the digoxigenin-responsive antibody switch. A two step process began by (FIG. 17A) functionalizing a portion of the scaffold strand with the blocking strand (in this case a digoxigenin molecule) through amine-reactive crosslinking chemistry to form the blocking analyte DNA. This modification was validated through mass spectroscopy. The complete scaffold and blocking strand was then assembled (FIG. 17B) through splinted ligation of the blocking analyte DNA to a click handle DNA which bears a DBCO moiety for later conjugation to the antibody. This ligation was validated through gel electrophoresis.

### Electrochemical Implementation of Biotin Antibody Switch

FIG. 8 shows a schematic of an electrochemical readout scheme for sensing using the antibody switch by incorporating redox reporting moieties conjugated onto the antibody (detecting strand). Data for real-time measurement using this system showed target-responsiveness through electrochemical readout.

### Monolithic Dual Antibody Clamp (MDAC): A High Avidity FRET Construct for Instant ELISA

### Introduction

Protein detection is of great importance to clinical diagnostics as it is one of the predominant methods of detecting and monitoring disease states. Protein quantitation in blood samples is typically achieved through ELISA assays. Due to the sample preparation and assay protocols, these assays are typically performed in off-site or centralized analytical laboratories by specialized technicians—this involves lengthy turnaround times that can impact patient outcomes. Current and past research into point-of-care analytical systems seeks to address the need for sensitive, rapid, and sample-prep-free protein detection and quantitation that can be carried out in the clinical setting, thus reducing sample-to-answer times.

Certain proposed methods address these needs by adopting analyte-specific detection approaches, such as enzymatic sensors, which are not readily generalizable to a plurality of targets. Other approaches, such as PLA (proximity ligation) and ECPA (electrochemical proximity assay), leverage the generality of immunosorbent assays in homogeneous assays, yet still require sample processing, lengthy incubations and/or addition of reagents. Ideally, a point-of-care protein detection system would require the simple addition of a blood draw to a benchtop instrument which achieves rapid quantitation at relevant endogenous target concentrations.

Here, we propose a new approach to protein quantification which preserves the generality of immunosorbent techniques in an assay free of sample handling or processing steps. This is achieved with a monolithic dual antibody ‘clamp’ (MDAC) construct, an embodiment of a double-antibody construct, conjoined by a DNA scaffold. When the two fluorophore-labeled monoclonal antibodies bind and ‘clamp down’ on a single target protein, their proximity leads to a FRET signal change. Excitation and probing of the MDAC is achieved by surface coupling with a tapered fiber optic (fiber-MDAC): an evanescent excitation field probes the binding state of MDAC constructs and the FRET emission response couples back into the fiber for optical detection. The fiber optic tip is directly immersed in a blood draw sample and quantitation is achieved within minutes. The fiber could be easily disposed and replaced for subsequent measurements. The platform is adaptable to small protein targets with at least two binding domains, which includes any target for which a traditional sandwich ELISA assay exists, holding great promise as a new rapid immunosorbent analytical technique for the clinical environment.

### Results and Discussion

### The MDAC Construct is a Monolithic Sandwich ELISA

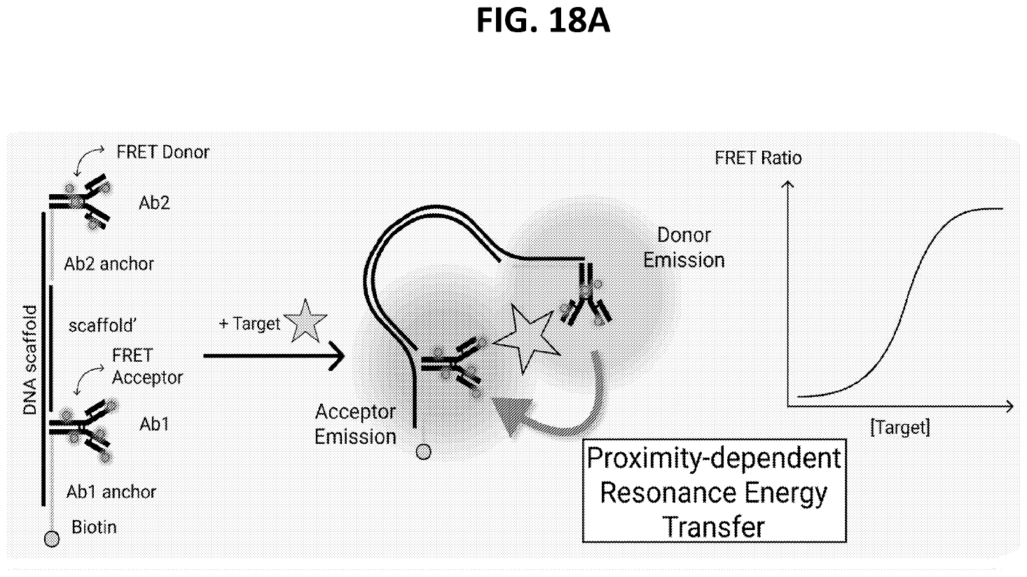

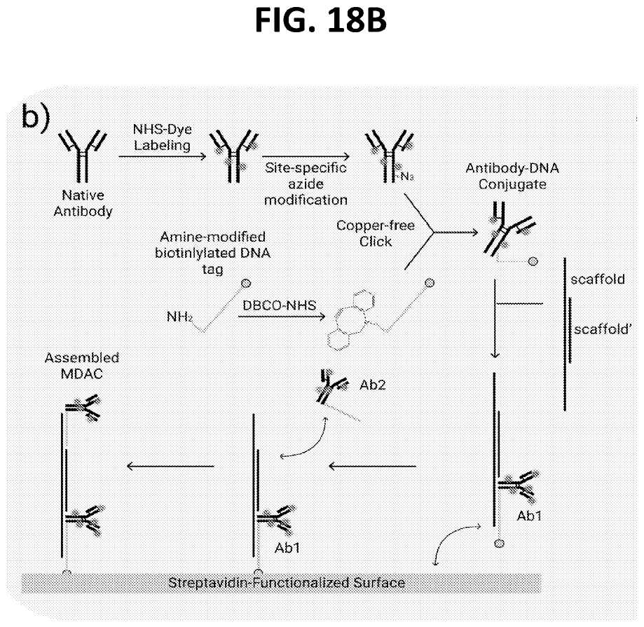

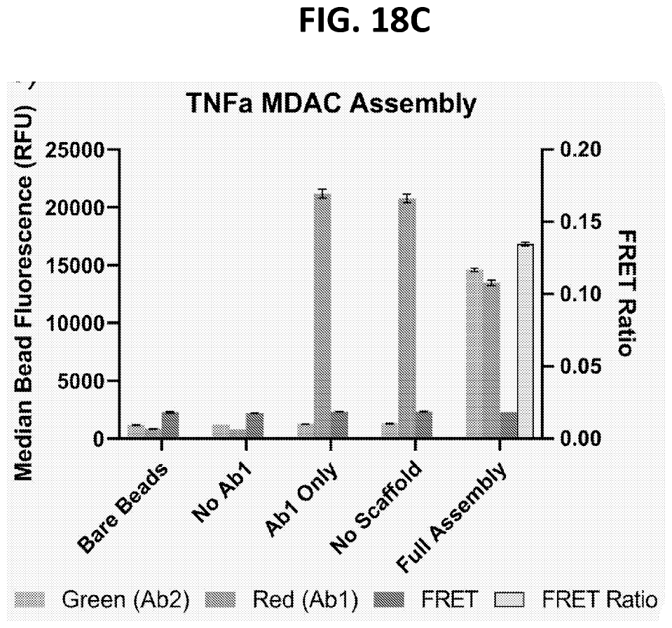

The MDAC construct consists of two fluorophore-labeled monoclonal antibodies linked by a DNA scaffold (FIG. 18A). In the presence of target, the antibodies will bind to two separate epitopes of the same protein, leading to an increase in proximity of the fluorescent dyes and efficiency of energy transfer between then. If the donor dye is excited with monochromatic light, in the presence of target the emission intensity of the acceptor dye will increase while that of the donor dye will decrease. The ratio of emission intensities is proportional to the fraction of bound MDACs.

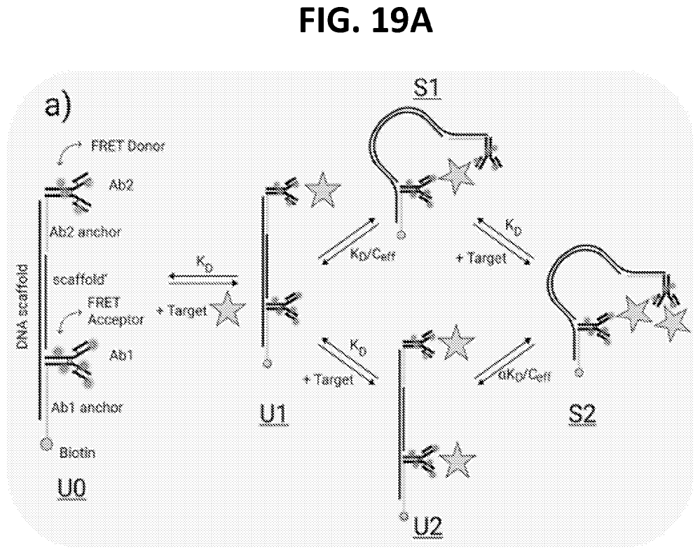

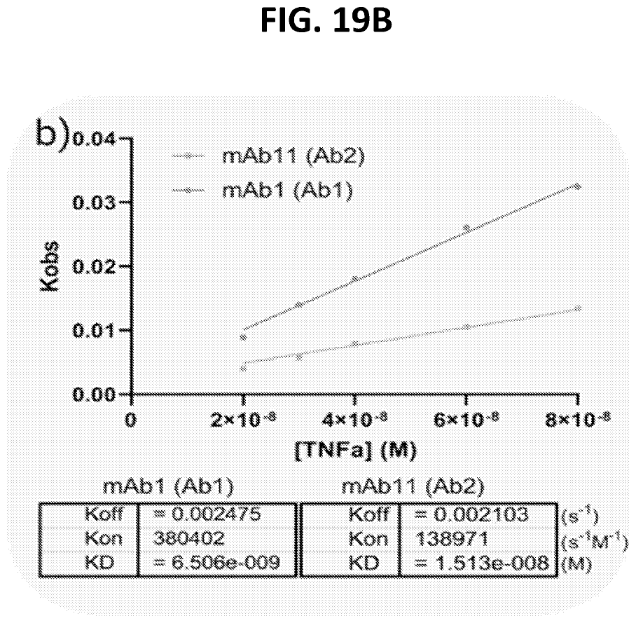

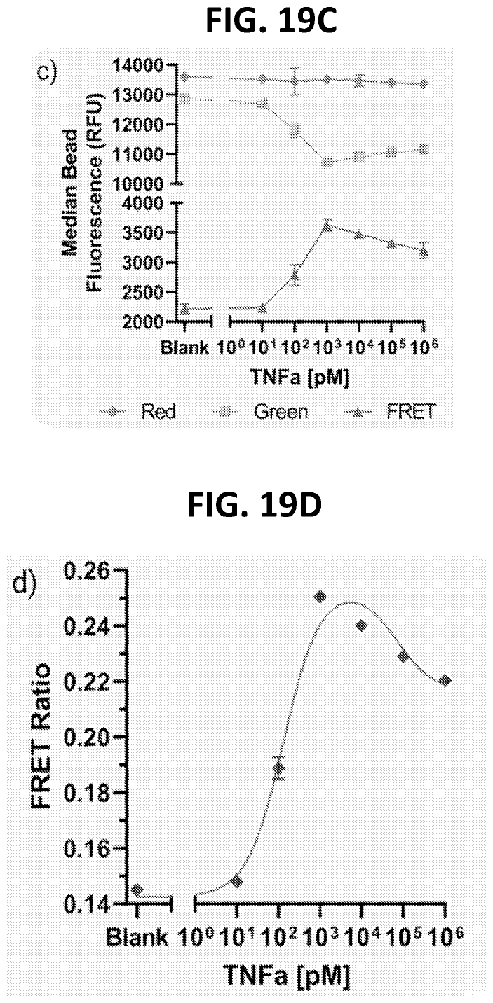

First, we assembled MDAC on streptavidin beads (FIGS. 19B and 19C). We chose two monoclonal anti-TNFa antibodies from the literature based on their competency for sandwich formation. We began by fluorescently labeling the two native antibodies. Clone mAb1 was labeled with Alexa Fluor 647 (red), while clone mAb11 was labeled with Alexa Fluor 546 (green)—the dyes were chosen for their comparatively high brightness and large Forster radius. The dye coupling was realized via amine-reactive NETS-ester dyes. We evaluated the degree of labeling via spectrophotometry, and the reaction was titrated versus dye concentration to obtain 4-6 dyes per antibody. Next, we modified the carbohydrate domain of the dye-labeled antibodies to incorporate an azide group. Separately, we DBCO-labeled a synthetic oligonucleotide, termed Ab1-anchor, with a 3′ biotin and a 5′ amine, via amine-reactive NHS-ester-DBCO. Another 5′ amine modified oligonucleotide, termed Ab2-anchor, was similarly modified with DBCO, and we assessed the degree of DBCO labeling via spectrophotometry. We conjugated the Ab1-anchor with azide-modified mAb1 via copper-free click chemistry. Similarly, Ab2-anchor was conjugated with azide-modified mAb11. We verified antibody-oligo conjugation by spectrophotometry and denaturing PAGE. Next, the MDAC scaffold and scaffold' oligos were slowly annealed on a thermocycler. We added the annealed duplex to Ab1 in excess and the hybridization reaction was incubated overnight. To achieve suitable spacing of the MDAC constructs immobilized on streptavidin beads, we pre-incubated the beads with a biotin solution to reduce the density of biotin binding sites prior to incubation with the Ab1-scaffold construct. We then washed the beads and incubated with Ab2 for the final hybridization step. After a final wash, MDAC beads were stored at 4 C.

After each assembly step we monitored the fluorescence of the beads with a flow cytometer (FIG. 18C). We saw a 16-fold increase in the red channel after incubation with the Ab1-scaffold construct. Similarly, we saw a 12× increase in green fluorescence upon subsequent incubation with Ab2. The latter signal increase was conditional on the presence of the DNA scaffold as well as Ab1, indicating negligible non-specific binding of Ab2 and scaffold DNA to the bead surface. We further validated the MDAC using single-molecule TIRF experiments, to verify the co-localization of the two antibodies onto the scaffold.

### We Modeled the Thermodynamics of MDAC Binding with a 5-State Model

The proximity of the two scaffold-bound antibodies warrants advantageous binding properties, among which is a strong avidity for the target. The binding behavior is approximated by a 5-state model (FIG. 19A). State U0 describes MDAC in its unbound conformation in the absence of target—in state U0, the mean distance of the antibodies is sufficient to generate negligible FRET. When target is present, it will first bind to Ab1 or Ab2—this is shown in state U1. Equilibrium between U0 and U1 is dependent on target concentration and antibody affinity, which we approximate with a single dissociation constant, KD,Ab. At low to moderate target concentrations, the MDAC conformation will readily switch to state S1, where both antibodies are bound to the target. The equilibrium between U1 and S1 is largely independent of target concentration, as the effective concentration of the target with respect to the second binding event is set by the volume swept by the scaffold-linked antibody and its corresponding entropic state space. We model this behavior with an avidity factor, Ceff. State S1 is FRET-compentent and thus leads to a binding signal. At higher target concentrations, two different targets may bind to MDAC—this is described by state U2. In this state, the dyes do not undergo FRET, and thus the structure does not produce a signal. Thus, we expect a decrease in MDAC signal at high concentrations, resembling that associated with the hook effect in typical immunoassays. The equilibrium between U1 and U2 is once again set by target concentration and KD,Ab. Due to the existence of two antigen-binding regions on each antibody, the two targets in state U2 may be bound to the antibodies in ways that are unfavorable to further conformational switching.

In some cases, however, one of the two targets may be shared between the antibodies. This leads to FRET-competent state S2. The presence of two targets will affect the entropic state space associated with intramolecular binding—we model this difference with an adjustment factor α for Ceff. Boltzmann weights corresponding to the states described above are reported in Table 1, and the resulting expression for the total FRET-competent partition is given by,

$$FRET = \frac{4 \frac{[ T ] {C}_{eff}}{{K}_{D , Ab}^{2}} + 4 \frac{{[ T ]}^{2} {C}_{eff}}{α {K}_{D , Ab}^{3}}}{1 + 4 \frac{[ T ]}{{K}_{D , Ab}} + 4 \frac{{[ T ]}^{2}}{{K}_{D , Ab}^{2}} + 4 \frac{[ T ] {C}_{eff}}{{K}_{D , Ab}^{2}} + 4 \frac{{[ T ]}^{2} {C}_{eff}}{α {K}_{D , Ab}^{3}}} . \qquad ( Eq . 1 )$$

TABLE 1
Summary of Boltzmann Weights for 5-State Binding Model.

| State | Boltzmann Weight |
|---|---|
| U0 | 1 |
| U1 | $4 \frac{[ T ]}{{K}_{D , Ab}}$ |
| U2 | $4 \frac{{[ T ]}^{2}}{{K}_{D , Ab}^{2}}$ |
| S1 | $4 \frac{[ T ] {C}_{eff}}{{K}_{D , Ab}^{2}}$ |
| S2 | $4 \frac{{[ T ]}^{2} {C}_{eff}}{α {K}_{D , Ab}^{3}}$ |

Boltzmann weight multiplicities are due to the existence of two Fab regions on each antibody, which increases the possible binding configurations. For sake of simplicity, we chose to exclude further binding configurations, such as those involving three or more targets and those involving more than one shared target between the antibodies. These configurations will tend to appear at exceedingly high target concentrations or are expected to be comparatively unfavorable at the observed concentrations.

We validated the binding model experimentally by first measuring the affinity of clones mAb1 and mAb11 via biolayer interferometry (BLI) (FIG. 19B). Observed on-rate, k_{obs}, for different target concentrations were obtained through exponential fits to the BLI sensor association curves. The off-rate, k_{off}, of each antibody was obtained as the average measured off-rate across all concentrations. On-rate of each antibody was then obtained (FIG. 19B) as the slope of the best-fit line with equation:

k_{obs}=kₒₙ·T+k_{off}. (Eq. 2)

The dissociation constants, K_{D}, of the antibodies were then computed via the definition

$${K}_{D} = \frac{{k}_{off}}{{k}_{on}} .$$

These were found to be 6.5 nM and 15.1 nM for mAb1 and mAb11, respectively. Next, we challenged MDAC beads with a wide range of unlabeled TNFa concentrations, allowed the structure to reach equilibrium overnight, and measured donor and FRET signals on a cytometer (FIG. 19C). Donor fluorescence sharply decreased and FRET intensity increased over the concentration range 10 pM-1 nM. At higher concentrations, the MDAC signal decreased due to the onset of state U2, as previously described. The FRET ratio was computed as FRET/(FRET+donor). Then, using GraphPad Prism, the ratio was fit to Eq. 1, modified with a scaling pre-factor and an additive background term (FIG. 19D). The geometric mean of mAb1 and mAb11 K_{D} was used for K_{D,Ab}. The model was found to fit the data well—a value of 180 nM was found for C_{eff} and a value of 10.3 for α. We define an operational dissociation constant, K_{D,Op}, as [T] at 50% of maximum signal, and find K_{D,Op}=120 pM, which is approximately 100× fold lower than the K_{D,Ab}, indicating that the MDAC structure features higher avidity than the individual antibodies. We also define the limit of detection (LOD), as [T] at which signal is equal to background plus 3 standard deviations. This parameter is equal to approximately 10 pM for MDAC deployed on this bead system.

### Bead MDAC can Perform Rapid Protein Quantitation in Serum

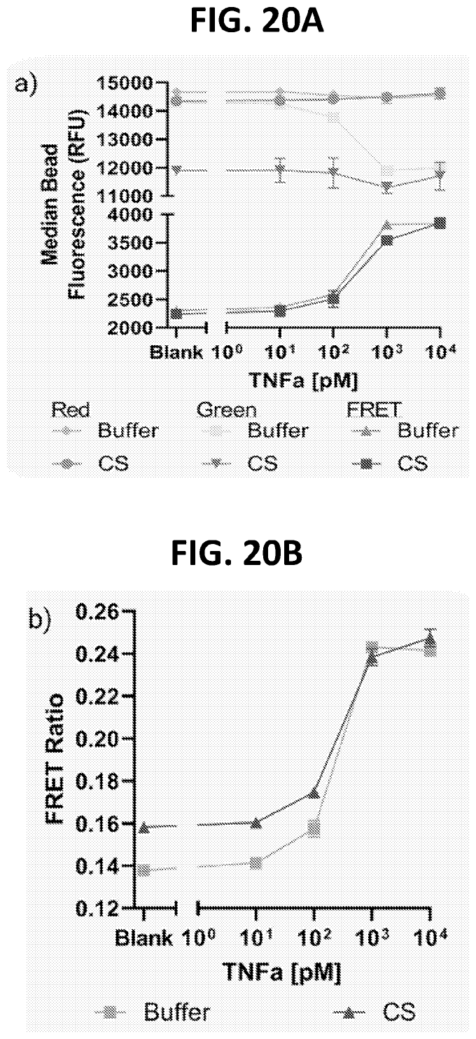

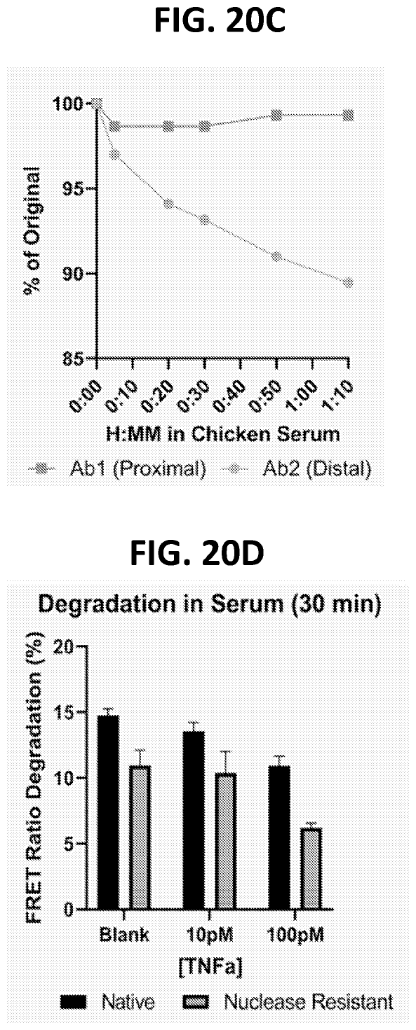

Next, we tested the feasibility of using MDAC to perform rapid quantitation of target concentrations directly in serum. MDAC constructs were assembled on beads and dispensed in chicken serum (CS) titrated with a range of TNFa concentrations. The beads were incubated for 30 minutes, magnetically separated from serum, washed, and resuspended in buffer. The procedure was repeated with TNFa in buffer as control. The beads were finally imaged on a cytometer to measure donor and acceptor fluorescence (FIG. 20A).

The data shows a significant increase in FRET fluorescence and decrease in green donor fluorescence between 100 pM and 1 nM, indicating that the binding kinetics of this MDAC structure are sufficiently rapid to carry out a measurement in only 30 minutes. For MDAC deployed in serum, we also note a significant decrease in green fluorescence at lower concentrations. This is attributed to the activity of nucleases in the serum that degrade the MDAC DNA scaffold, leading to the loss of the distal green antibody. The decrease in green donor fluorescence at low concentrations leads to an increase in FRET ratio background (FIG. 20B). The proximal red antibody, however, experienced minimal degradation, indicating that nuclease activity on the short DNA portion anchoring the structure to the bead is small compared to that affecting the central portion of the DNA scaffold. Sterics from the bead surface account for some of this disparity as well. Sterics are likely the explanation for the reduced linker degradation at higher target concentrations—when the two antibodies are bound to the same target protein and are thus in proximity, the DNA scaffold is closer to the surface and less susceptible to nuclease degradation.

To further confirm that nuclease activity was the source of this signal degradation, we deployed the MDAC beads in target-free serum and characterized degradation of the structure over the course of 70 minutes (FIG. 20C). As expected, we recorded a steady decline in the distal green antibody and little change in the proximal antibody fluorescence. Although reducing serum incubation time to less than 30 minutes would reduce structural degradation, it would also decrease the amount of binding signal obtained. Conversely, incubating longer leads to marginally more binding signal, but significantly more degradation. Experimentally, we found that at low concentrations of TNFa, 30-minute incubations led to better SNR than 15 minute and 1 h incubations. An alternative strategy to mitigating nuclease activity was also explored. The phosphodiester bonds of the DNA scaffold were substituted with phosphorothioates in 20% of locations. The DNA anchor of the distal antibody was also modified with interspersed locked nucleic acid (LNA) bases. Both such synthetic DNA alterations have been shown to reduce susceptibility to nuclease activity. Serum degradation of this nuclease-resistant variant of MDAC was recorded at low TNFa concentrations, where the native MDAC structure is most susceptible to nuclease activity (FIG. 20D), and found that the synthetic DNA MDAC experienced reduced degradation, as expected. Future iterations of MDAC could leverage different synthetic polymers such as L-DNA to fully eliminate the impact of nuclease activity.

### MDAC is a General Platform—Target Protein Specificity Depends on the Selected Antibodies

To explore the generalizability of the platform towards different targets, a new MDAC structure was synthesized for MCP-1. For this iteration of the MDAC structure, Alexa Fluor 647 was substituted with Atto 643—although these two dyes are spectrally similar, Atto 643 has significantly higher quantum efficiency. Previous literature has shown that a high FRET acceptor quantum yield is of key importance to improved FRET efficiency, as a larger portion of the energy transferred from the donor dye is converted to observable fluorescence. Commercial vendors have reported that antibody clone 5D3-F7 forms sandwich pairs on MCP-1 targets both with clone 10F7 and clone 2H5. Bead MDAC structures were assembled with both pairs and preliminary binding curves in buffer showed that the 5D3-F7/10F7 structure resulted in larger FRET ratio signals, likely due to a more favorable placement of binding pockets—thus we proceeded with these clones for subsequent experiments.

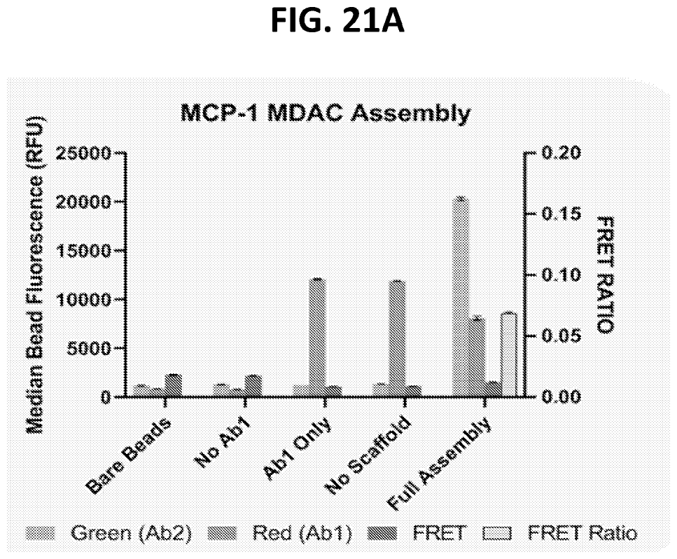

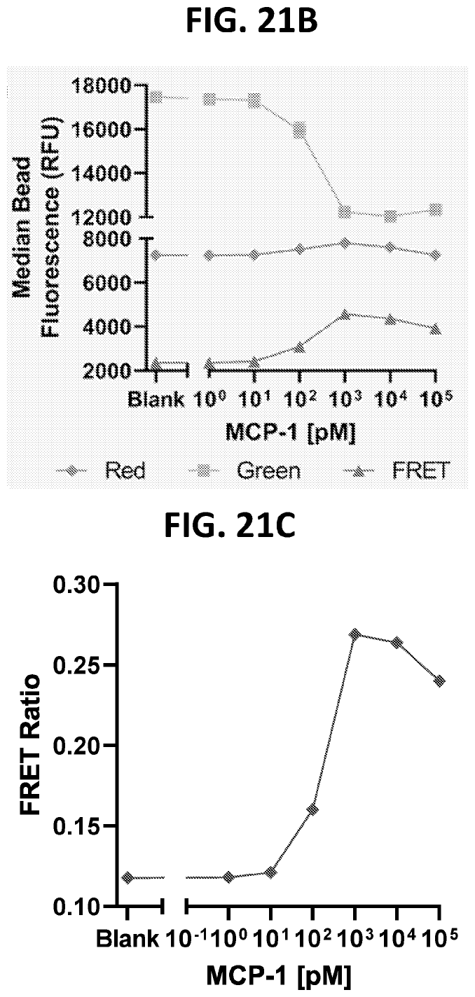

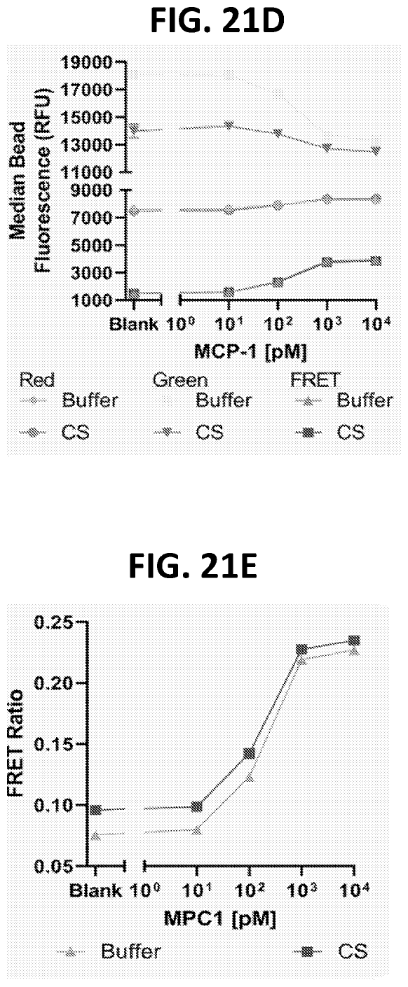

As a first step, the assembly of the MCP-1 MDAC on beads was characterized, with controls for non-specific assembly with the new antibody clones (FIG. 21A). As with the TNFa MDAC, the assembly was highly specific and reproducible. The intensity of red fluorescence was lower compared to TNFa MDAC due to the replacement of Alexa 647 with Atto 643. Next, kinetics and thermodynamics of the MCP-1 antibodies were characterized by BLI, as was done previously. We then challenged MDAC beads with a wide range of MCP-1 concentrations, allowed the structure to reach binding equilibrium overnight, and measured donor and FRET signals on a cytometer (FIG. 21B). Green donor fluorescence decreased, and FRET acceptor fluorescence increased over the MCP-1 concentration range 10 pM-1 nM. Beyond this range, the binding signal decreased, as with the TNFa MDAC. The FRET ratio was computed from this data and the Eq. 1 was fit to the data using the geometric mean of the two antibody KDs (FIG. 21C). Unsurprisingly, these values are not too dissimilar to those of the TNFa MDAC, since the geometry of the scaffold is conserved. Interestingly, the FRET ratio binding signal of the MCP MDAC was significantly larger than that of the TNFa structure. This can be attributed to the improved quantum yield of the acceptor dye, and a smaller target protein (MCP-1 molecular weight: ˜12 kDa, TNFa molecular weight: ˜17 kDa), which likely results in higher proximity of the antibodies when bound to the same target and thus more efficient resonant energy transfer.

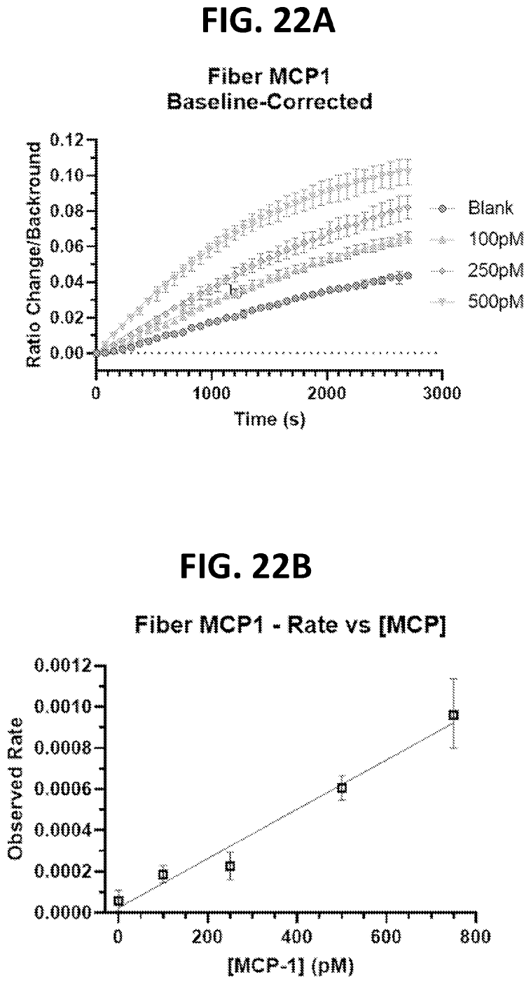

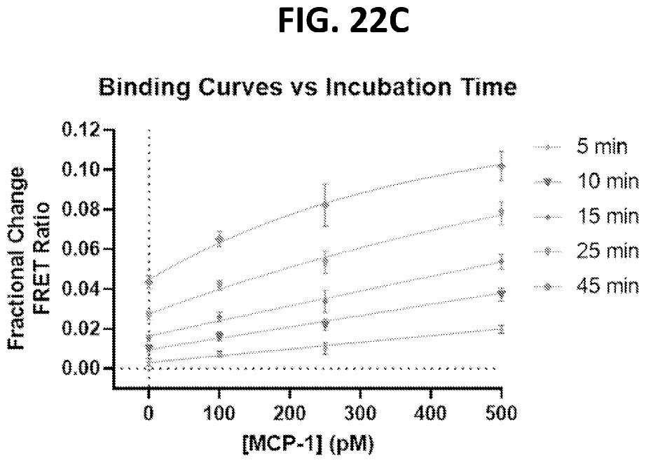

Next, we verified binding performance of the bead-immobilized structure in CS doped with MCP-1 concentrations, with a limited incubation time of 30 minutes (FIGS. 22D and 22E). As with the TNFa MDAC, the structure was found to generate a slightly reduced binding signal compared to the overnight incubation. Interestingly, the dynamic range of the measurement was found to be slightly expanded by the shortened incubation at the highest 10 nM concentration—this is possibly due to slower on-rate kinetics of state U2 (the state responsible for the decrease in signal at high concentrations) compared to bound-and-switched state S1. As before, we note a decrease in distal (green) antibody fluorescence due to nuclease activity that mostly impacts measurements at small target concentrations. This results in a small increase in background FRET ratio values, but the measurement in CS still features a large signal increase in the desired concentration range.

### MCP-1 Binding Response on Optical Fiber

Optical fiber surfaces were prepared with MCP-1 MDAC and exposed to different concentrations of MCP-1 in buffer (FIG. 22A). The MDAC probe was interrogated through the optical fiber and the fractional FRET ratio change compared to background was monitored for 45 minutes. Results show clearly discernible binding kinetics in the relevant concentration range. Error bars represent standard deviation of N>3 measurements. Exponential curves were fit to the data in and the rates were plotted vs MCP-1 concentration (FIG. 22B). A linear fit was computed for this kinetic data. Error bars represent 95% confidence intervals. To explore the feasibility of further reducing incubation time, data in FIG. 22A was re-organized and plotted vs concentration for different incubation times (FIG. 22C). This plot demonstrates the feasibility of distinguishing concentrations as low as 100 pM MCP-1 in as little as 15 minutes. Error bars represent 95% confidence intervals.

### MDAC is Employed in an Instant ELISA Platform for Sample Preparation-Free Protein Quantitation

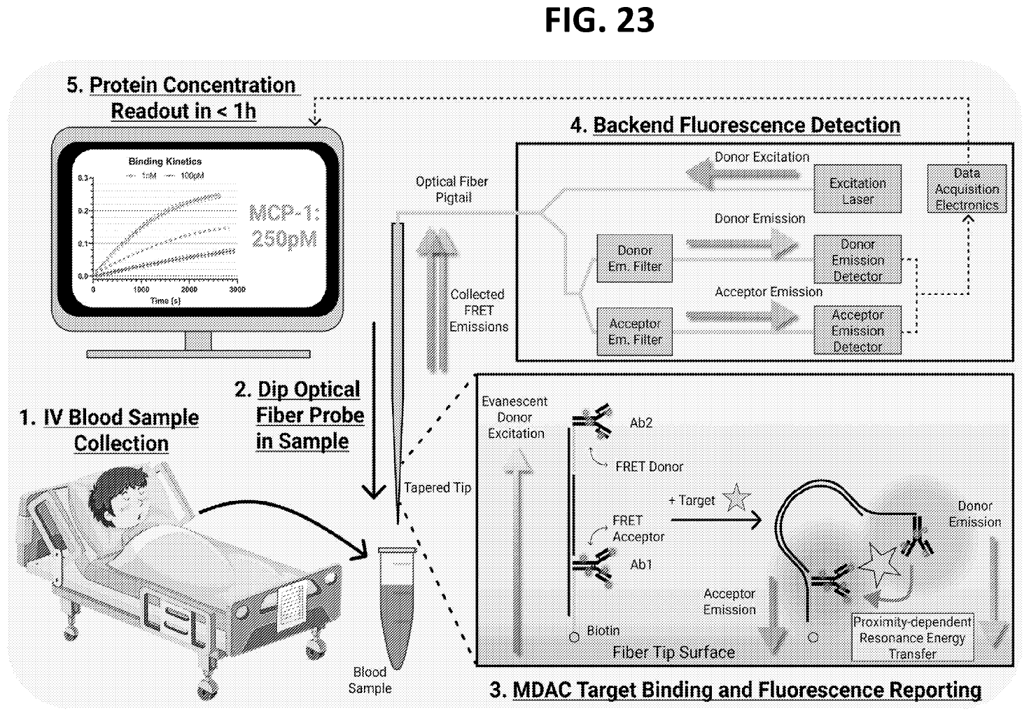

The MDAC is employed in an instant ELISA system through an optical fiber backend (FIG. 23). The construct is anchored to a streptavidin-functionalized optical fiber tip via a biotin label on the DNA. The tip is sharply tapered, such that excitation light carried through the fiber will excite the donor dye via an evanescent field. Venous blood is collected from the patient and the optical fiber is dipped in the sample, thus exposing MDAC to target. As the MDAC binds to target, dye emission fluorescence is collected back into the fiber and carried to the detection backend. Due to the exponential profile of the evanescent excitation field, auto-fluorescent molecules in the blood sample are not excited, leading to very limited background compared to epifluorescence imaging modalities. In the detection backend, collected emission is split between two filter-detector assemblies that measure emission intensity from both donor and acceptor dyes. Data acquisition electronics compute the emission ratio, thus monitoring the binding rate of MDAC molecules. The measured binding rate is compared to MDAC binding kinetics to determine the target concentration in the patient sample. The instant ELISA platform can accurately measure MCP-1 concentrations between 100 pM and 1 nM in less than 1 h since sample collection.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. An antibody construct comprising:
an antibody or a binding fragment thereof comprising a first label, wherein the antibody or the binding fragment thereof is linked to a blocking analyte and a second label via a linker,
wherein:
in the absence of a target analyte, the blocking analyte binds to the antibody or the binding fragment thereof, and
in the presence of the target analyte, the target analyte competes with the blocking analyte for binding to the antibody or the binding fragment thereof, and
the first and second labels interact to generate a detectable readout that changes depending on whether the blocking analyte is bound by the antibody or the binding fragment thereof, or is not bound by the antibody or the binding fragment thereof.

2. The antibody construct of claim 1, wherein:
(a) the second label is conjugated to the linker, or
(b) the second label is conjugated to the blocking analyte.

3. (canceled)

4. The antibody construct of claim 1, wherein:
(a) the first label is a fluorophore and the second label is a quencher; or
(b) the first label is a quencher and the second label is a fluorophore; or
(c) the first label is a donor fluorophore and the second label is an acceptor fluorophore; or
(d) the first label is an acceptor fluorophore and the second label is a donor fluorophore.

5-7. (canceled)

8. The antibody construct of claim 1, wherein the detectable readout is an optical signal, an electrical signal, an electrochemical signal, a nuclear magnetic resonance signal, or a biological signal.

9. (canceled)

10. The antibody construct of claim 1, wherein binding of the target analyte to the antibody or the binding fragment thereof increases or decreases the detectable readout.

11. An antibody construct comprising:
an antibody or a binding fragment thereof comprising a redox reporter, wherein the antibody or the binding fragment thereof is linked to a blocking analyte and a sensing electrode;
wherein:
in the absence of a target analyte, the blocking analyte binds to the antibody or the binding fragment thereof, and
in the presence of the target analyte, the target analyte competes with the blocking analyte for binding to the antibody or the binding fragment thereof, and
the redox reporter and the sensing electrode interact to generate an electrical signal that changes depending on whether the blocking analyte is bound by the antibody or the binding fragment thereof, or is not bound by the antibody or the binding fragment thereof.

12. The antibody construct of claim 11, wherein:
(a) the antibody or the binding fragment thereof is linked to the blocking analyte and the sensing electrode via a linker; or
(b) the antibody or the binding fragment thereof is linked to the blocking analyte by a first linker and the antibody or the binding fragment thereof is linked to the sensing electrode via a second linker; or
(c) the antibody or the binding fragment thereof is linked to the sensing electrode via a gold-thiol bond.

13-15. (canceled)

16. The antibody construct of claim 1, wherein the linker is:
(a) a double-stranded polynucleotide; or
(b) a single-stranded polynucleotide; or
(c) a partially double-stranded polynucleotide comprising at least one unpaired nucleotide; or
(d) a partially double-stranded polynucleotide, and wherein at least 1% of the length of the linker comprises unpaired nucleotide(s).

17-26. (canceled)

27. The antibody construct of claim 1, wherein:
(a) the blocking analyte and the target analyte are the same; or
(b) the blocking analyte is a structural analog of the target analyte.

28. (canceled)

29. The antibody construct of claim 1, wherein:
(a) a binding affinity of the antibody to the target analyte is between 90% and 110% of a binding affinity of the antibody to the blocking analyte; or
(b) the antibody has a higher binding affinity for the target analyte than for the blocking analyte; or
(c) the antibody has a lower binding affinity for the target analyte than for the blocking analyte.

33. (canceled)

34. An antibody construct comprising:
(a) two detecting strands, wherein a first detecting strand comprises a first antibody or a binding fragment thereof, and a second detecting strand comprises a second antibody or a binding fragment thereof; and
(b) a first label and a second label,
wherein the detecting strands or portions thereof are complementary and hybridize to each other or one or more scaffold strands, and
wherein in the presence of the target analyte, the first antibody and the second antibody bind to two different epitopes on the target analyte, and the first and second labels interact with each other to generate a detectable readout compared to when there is an absence of the target analyte.

35. The antibody construct of claim 34, wherein the two detecting strands hybridize to a scaffold strand, thereby linking the two detecting strands.

36. The antibody construct of claim 35, wherein:
(a) the two detecting strands hybridize to a single scaffold strand; or
(b) the two detecting strands hybridize to separate scaffold strands which scaffold strands hybridize to each other.

37. (canceled)

38. The antibody construct of claim 34, wherein:
(a) the two detecting strands hybridize to each other, thereby linking the two detecting strands; or
(b) the first label is linked to the first antibody and the second label is linked to the second antibody; or
(c) the first label and/or second label is linked to a scaffold strand.

39-43. (canceled)

44. The antibody construct of claim 3, wherein the antibody construct comprises two scaffold strands, wherein a first scaffold strand and a second scaffold strand are complementary and hybridize to each other.

45-47. (canceled)

48. The antibody construct of claims 4, wherein the second scaffold strand is at least 20 nucleotides long.

49-55. (canceled)

56. The antibody construct of claim 3, wherein the first antibody is covalently conjugated to the first detecting strand via an Fc region of the first antibody, and wherein the second antibody is covalently conjugated to the second detecting strand via an Fc region of the second antibody.

57-59. (canceled)

60. The antibody construct of claim 3, wherein the binding of the target analyte to the first and second antibodies increases or decreases the detectable readout.

61. A method of detecting a target analyte in a sample, comprising:
(1) contacting the sample with an antibody construct of claim 1; and
(2) measuring binding of the antibody construct to the target analyte using a detectable readout from the antibody construct.

62-64. (canceled)

65. A method of adjusting kinetics and/or effective binding affinity of an antibody construct, comprising:
(1) generating an antibody construct of claim 1;
(2) measuring binding of the antibody construct to a target analyte;
(3) changing one or more components of the antibody construct;
(4) re-measure binding of the antibody construct to the target analyte; and
(5) optionally repeat steps (3) and (4) until the desired kinetics and/or effective binding affinity of the antibody construct is reached.

66-72. (canceled)
